(19) United States
(12) Patent Application Publication
(10) Pub. No.: US 2024/0138839 A1
(43) Pub. Date: May 2, 2024

(54) SYSTEMS AND METHODS FOR PRESERVING AND MANIPULATING OF ACUTE OTOMIES

(71) Applicant: G.I. Windows, Inc., Westwood, MA (US)

(72) Inventors: Dane T. Seddon, Boston, MA (US); Jonathan P. Boduch, Quincy, MA (US); Michael Boutillette, Westwood, MA (US); Dana Zitnick, Westwood, MA (US); Marvin Ryou, Lynnfield, MA (US)

(21) Appl. No.: 18/384,022

(22) Filed: Oct. 26, 2023

Related U.S. Application Data
(60) Provisional application No. 63/435,724, filed on Dec. 28, 2022.
(60) Provisional application No. 63/419,509, filed on Oct. 26, 2022.

(51) Int. Cl. A61B 17/11

(52) U.S. Cl. CPC A61B 17/1114; A61B 2017/00867

(57) ABSTRACT

Various types of otomy control devices that secure a periphery and/or adjacent tissue of a target otomy site and maintain the size of an otomy through a single wall of a vessel, organ, or lumen within the body. Among other things, this can reduce unintentional trauma, dilation, contraction, or locomotion of an otomy during a surgical procedure. They also can serve a beneficial purpose of allowing immediate communication between lumens before the complete creation of A permanent anastomosis. In some embodiments, magnetic compression anastomosis devices and/or other devices can be deployed through a channel of the otomy control device such as for deployment in a distal lumen.

### CROSS REFERENCE TO RELATED APPLICATIONS

This patent application claims the benefit of U.S. Provisional Patent Application No. 63/435,724 entitled SYSTEMS AND METHODS FOR PRESERVING AND MANIPULATING OF ACUTE OTOMIES filed Dec. 28, 2022 and U.S. Provisional Patent Application No. 63/419,509 entitled SYSTEMS AND METHODS FOR PRESERVING AND MANIPULATING OF ACUTE OTOMIES filed Oct. 26, 2022, each of which is hereby incorporated by reference in its entirety.

### FIELD OF INVENTION

The invention relates to devices and methods for preserving, manipulating, and capturing acute otomies, specifically intraoperative capture of acute otomies between two or more compression anastomosis devices.

### BACKGROUND

Bypasses or diversions of the gastroenterological (GI), cardiovascular, or urological systems traditionally were often formed by cutting holes (i.e., acute otomies) in tissues at two locations and joining the holes with sutures or staples. A bypass or diversion is typically placed to route fluids (e.g., secretions, blood, nutrients) between healthier portions of the system, while bypassing diseases or malfunctioning tissues or to shorten the overall pathway of a lumen system. The procedure is typically invasive, and subjects a patient to risks such as bleeding, infection, pain, and adverse reaction to anesthesia. Additionally, a bypass or diversion created with sutures or staples can be complicated by post-operative leaks and adhesions. Leaks may result in infection or sepsis, while adhesions can result in complications such as bowel strangulation and obstruction. While traditional bypass procedures can be completed with an endoscope, laparoscope, or robot, it can be time consuming to join the holes cut into the tissues. Furthermore, such procedures require specialized expertise and equipment that are not available at many surgical facilities.

As an alternative to sutures or staples, surgeons can use mechanical couplings or magnets to create a compressive anastomosis between tissues. For example, compressive couplings or paired magnets can be delivered to tissues to be joined. Because of the strong compression, the tissue trapped between the couplings or magnets is cut off from its blood supply. Under these conditions, the tissue becomes necrotic and degenerates, and at the same time, new tissue grows around points of compression, e.g., on the edges of the coupling. With time, the coupling can be removed, leaving a healed anastomosis between the tissues.

Current practices for accessing internal lumens/organs to deliver compressive couplings or magnets often require the creation of acute otomies. Upon completion of the desired surgery, these acute otomies require subsequent closure procedures. This adds procedural steps, increases procedure time, and requires effort on the part of the physician to preserve the otomy against tearing/expansion due to intraoperative manipulation. This also adds risk for the patient in the form of uncontrolled tearing/expansion of the otomies due to manipulation resulting in acute or even chronic anastomotic leaks. Additionally, compression anastomoses may take up to 2 weeks to develop, which prevents immediate positive effects of the procedure and may limit the types of procedures that can be performed with compressive anastomotic devices.

Thus, there remains a need for devices and method that reduce the need for otomy closure steps and create immediate communication between lumens before the complete creation of the permanent anastomosis.

### SUMMARY OF VARIOUS EMBODIMENTS

In accordance with one embodiment of the invention, an otomy control device comprises a distal flange and a proximal flange separated and connected by a central channel, wherein the distal flange and the proximal flange are configured to compress to fit the otomy control device within a working channel of an access or delivery device in a delivery configuration and to expand on opposite sides of an otomy to secure the otomy when delivered from the working channel in a fully deployed configuration.

In various alternative embodiments, each flange may comprise a shape-memory material such as a nitinol shape-memory material or a spring material. Each flange may comprise an inner framework and an external covering encompassing some or all of the inner framework, where the external covering may include a biocompatible material and may include a flexible material such as plastic, rubber, urethane, or polymer which may be formed on or over the inner framework such as by injection molding. Alternatively, the distal flange, the proximal flange, and the channel may be formed as a unitary device. The flanges may be inflatable. The device may include one or more magnets, e.g., situated in the distal flange, situated in the proximal flange, and/or situated about the central channel. At least one of the flanges may be shaped or configured to assist with positioning of a magnetic compression anastomosis device around the otomy control device, e.g., tapered. The flanges may have the same configuration or may have different configurations. The proximal flange may be shaped or configured to mate with a proximal flange of another otomy control device. The otomy control device may be a two-piece device comprising a distal half including the distal flange and a proximal half including the proximal flange, wherein the distal half and the proximal half are configured to interlock to form the central channel, in which case the device may include a locking mechanism for drawing the distal and proximal halves into proximity and/or preventing the distal and proximal halves from separating after being brought into proximity. The otomy control device may include at least one connecting member to assist with mating of the device with another otomy control device. In various alternative embodiments, each flange may include one or more loops or lobes.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows an exemplary apparatus for controlling an acute otomy.

FIGS. 2A-2I schematically show the procedural steps for creation, control, and healing of an acute otomy.

FIG. 3 schematically shows a silicone grommet identifying the intraluminal and extraluminal flanges and how the grommet sits transmurally in the tissue.

FIG. 4 schematically shows a nitinol tubing/wire hybrid grommet.

FIG. 5 schematically shows a nitinol tubing/wire hybrid grommet.

FIGS. 6A-6B schematically show a nitinol tubing/wire hybrid grommet.

FIGS. 7A-7B schematically show nitinol wire array clamps.

FIG. 8 schematically shows an inflatable grommet.

FIG. 9 schematically shows an apparatus for controlling an otomy.

FIG. 10 schematically shows an apparatus for controlling an otomy.

FIG. 11 schematically shows an apparatus for controlling an otomy.

FIG. 12 schematically shows a sprung clamping element for securing an otomy.

FIGS. 13A-13B schematically show perspective views of nitinol tubing apparatuses for securing an otomy.

FIGS. 14A-14B schematically show perspective views of nitinol tubing apparatuses for securing an otomy.

FIGS. 15A-15B schematically show perspective views of nitinol tubing apparatuses for securing an otomy.

FIGS. 16A-16B schematically show perspective views of nitinol tubing apparatuses for securing an otomy.

FIGS. 17A-17B schematically show perspective views of nitinol tubing apparatuses for securing an otomy.

FIGS. 18A-18B schematically show perspective views of nitinol tubing apparatuses for securing an otomy.

FIGS. 19A-19B schematically show perspective views of nitinol tubing apparatuses for securing an otomy.

FIGS. 20A-20B schematically show perspective views of nitinol tubing apparatuses for securing an otomy.

FIGS. 21A-21B schematically show perspective views of nitinol tubing apparatuses for securing an otomy.

FIGS. 22A-22B schematically show perspective views of nitinol tubing apparatuses for securing an otomy.

FIGS. 23A-23B schematically show perspective views of nitinol tubing apparatuses for securing an otomy.

FIGS. 24A-24B schematically show perspective views of nitinol tubing/wire hybrid apparatuses for securing an otomy.

FIGS. 25A-25B schematically show perspective views of nitinol tubing/wire hybrid apparatuses for securing an otomy.

FIG. 26 schematically shows a view of a nitinol tubing/wire hybrid apparatus for securing an otomy.

FIGS. 27A-27B schematically show perspective views of ratcheting securing apparatuses for securing an otomy.

FIGS. 28A-28B schematically show perspective views of a circular securing apparatus for securing an otomy.

FIGS. 29A-29B schematically show a stent-style nitinol array apparatus for securing an otomy.

FIGS. 30A-30B schematically show a stent-style nitinol array apparatus for securing an otomy.

FIGS. 31A-31B schematically show a stent-style nitinol array spiraled wire apparatus for securing an otomy.

FIGS. 32A-32B schematically show an inflatable grommet apparatus for securing an otomy.

FIGS. 33A-33B schematically show a two-piece suture clamp apparatus for securing an otomy.

FIGS. 34A-34B schematically show a snap lock apparatus for securing an otomy.

FIGS. 35A-35B schematically show a sliding arms otomy clip for securing an otomy.

FIG. 36 schematically shows a foam grommet apparatus for securing an otomy.

FIG. 37 schematically shows a clip secured grommet otomy clip apparatus for securing an otomy.

FIG. 38 schematically shows a coil compressed grommet apparatus for securing an otomy.

FIGS. 39A-39B schematically show perspective views of an otomy clip apparatus for securing an otomy.

FIGS. 40A-40D schematically show coiled wire concepts for securing an otomy.

FIG. 41 schematically shows an adhesive reinforced apparatus for securing an otomy.

FIGS. 42A-42B schematically show an otomy clip patch for securing an otomy.

FIGS. 43A-43B schematically show an otomy clip puncture patch for securing an otomy.

FIG. 44 schematically shows a crimped wire form otomy clip for securing an otomy.

FIGS. 45A-45O schematically show formation of a duodenum otomy for a single anastomosis duodenal ileostomy (SADI) with endo intracorporeal anastomosis (EIA) using an otomy control device and self-assembling magnetic compression anastomosis device, in accordance with certain embodiments.

FIGS. 46A-46G schematically show formation of an ileum otomy for a single anastomosis duodenal ileostomy (SADI) with endo intracorporeal anastomosis (EIA) using an otomy control device and self-assembling magnetic compression anastomosis device, in accordance with certain embodiments.

FIGS. 47A-47I schematically show formation of an anastomosis for a single anastomosis duodenal ileostomy (SADI) with endo intracorporeal anastomosis (EIA) using an otomy control device and self-assembling magnetic compression anastomosis device, in accordance with certain embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Exemplary embodiments of the invention include systems and methods for preserving and manipulating acute otomies. Various embodiments allow for intraoperative capturing of otomies between two or more devices to create a compression anastomosis. The embodiments described herein may reduce or eliminate the need for closure steps of the acute otomies, reducing the risk of anastomotic leaks. Certain embodiments of the present invention include various types of otomy control devices that secure a periphery and/or adjacent tissue of a target otomy site and maintain the size of an otomy through a single wall of a vessel, organ, or lumen within the body. Among other things, this can reduce unintentional trauma, dilation, contraction, or locomotion of an otomy during a surgical procedure. They also can serve a beneficial purpose of allowing immediate communication between lumens before the complete creation of the permanent anastomosis. In some embodiments, magnetic compression anastomosis devices and/or other devices can be deployed through a channel of the otomy control device such as for deployment in a distal lumen.

As used herein, distal and proximal shall be in reference to the access device handle. As used herein particularly with regard to an access or delivery device for deploying a magnetic compression anastomosis device or an otomy control device, the terms “distal” and “proximal” are generally relative to such access or delivery device, e.g., with the “distal” being further from the access or delivery device than the “proximal.” In some situations, the terms “distal” and “proximal” may be relative to a lumen, e.g., with the “distal” generally being within the lumen and the “proximal” generally being outside of the lumen.

It should be noted that certain embodiments are described herein with reference to a nitinol shape-memory material, although it should be noted that embodiments are not limited to nitinol but instead other shape-memory materials may be used in various alternative embodiments including, without limitation, a material that can act as a spring.

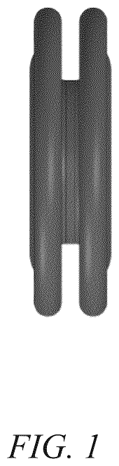

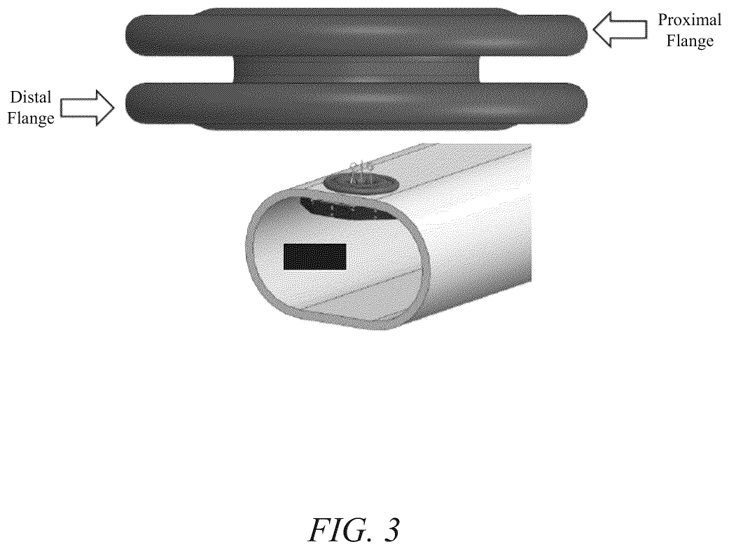

FIGS. 1-3 show an exemplary otomy control device in accordance with certain embodiments. As with many of the otomy control devices described herein, the otomy control device shown in FIGS. 1-3 is a dual-flange style apparatus (referred to herein as a grommet). As shown in FIG. 1 and FIG. 3, a grommet apparatus may include a distal flange and a proximal flange connected by a channel of smaller diameter than the flanges. The central channel between the flanges connects the flanges while capturing and controlling the size of the otomy. The flanges engage the tissue surrounding the otomy while the central channel connecting the flanges allows for fluidic passage through the grommet. The grommet is generally, although not necessarily, an annular shape.

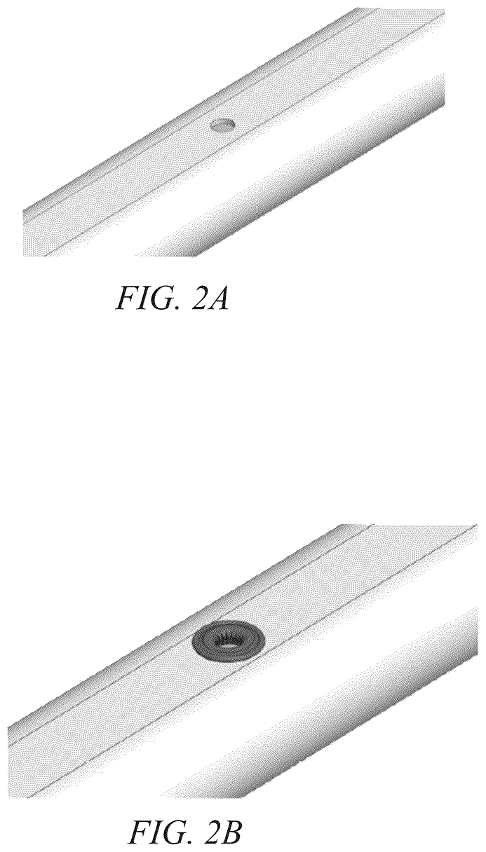

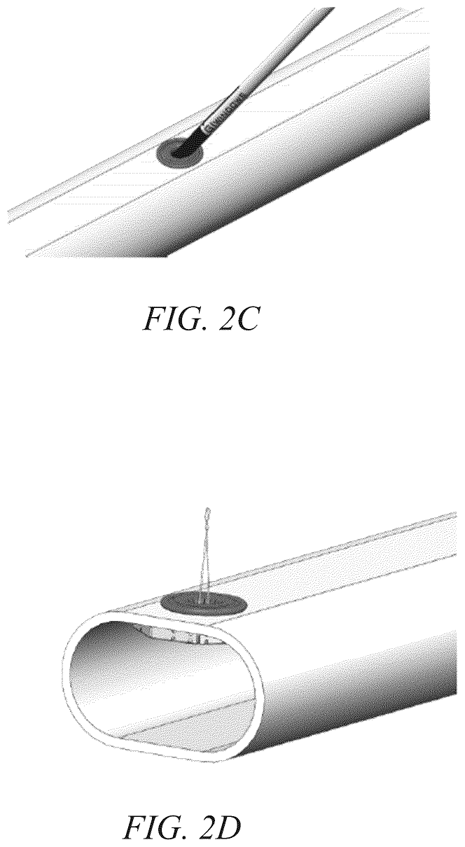

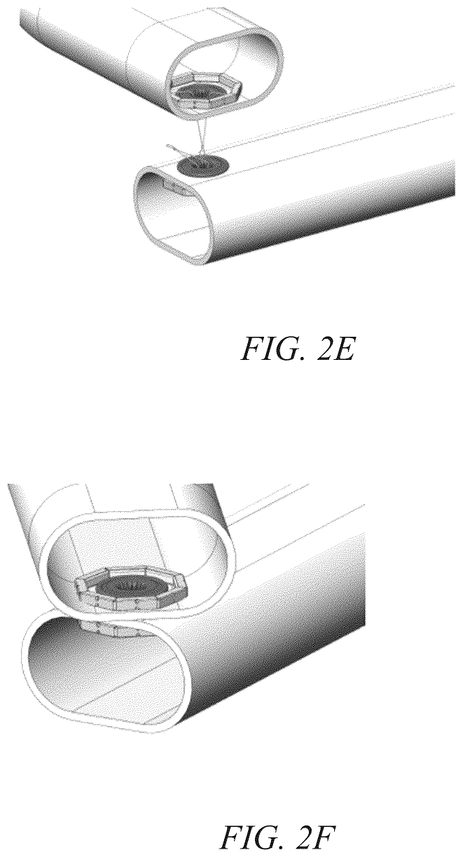

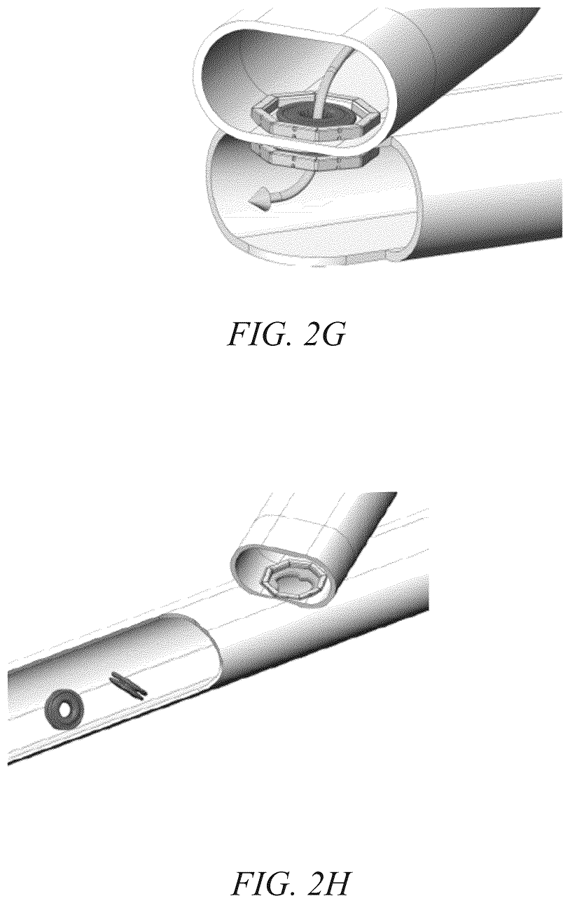

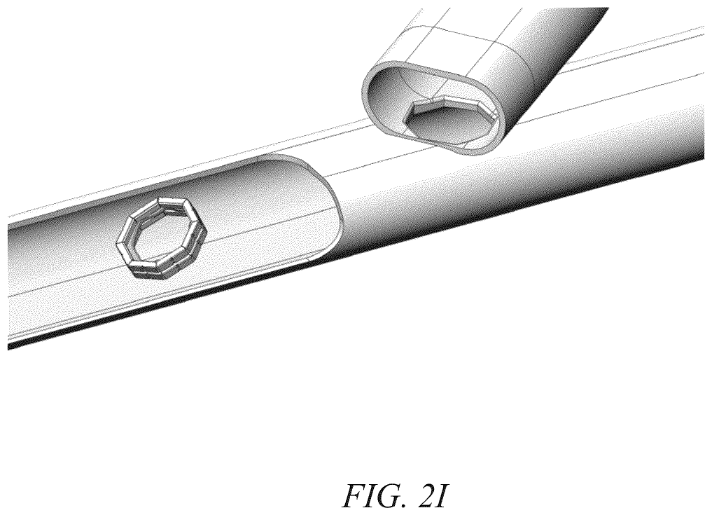

FIGS. 2A-2I show a method of controlling an otomy using an otomy control device of the type shown in FIG. 1 such as during the creation of a permanent anastomosis. Generally speaking, the procedure for controlling an otomy and anastomosis formation may be completed endoscopically, laparoscopically, robotically, by way of open-field surgery, or a combination thereof.

An otomy is formed in an organ and/or bowel as shown in FIG. 2A. After otomy formation, an access device cannulates the otomy, entering the distal side of the tissue into the organ/bowel lumen.

A grommet apparatus is compressed within a working channel of an access device in a delivery configuration, with the distal flange toward the distal end of the access device, and the proximal flange toward the proximal end of the access device. The access device may house the grommet within a rigid tube such as a cannula, or via a flexible tube such as a catheter or endoscope. The grommet may, in some embodiments, be housed within a working channel of a secondary delivery device within the access device. The secondary delivery device may move proximally, distally, and rotationally within the access device. The secondary delivery device may house the grommet prior to grommet deployment into the body of a patient.

The access device may, in some embodiments, comprise a pusher device, in addition to or in replacement of the secondary delivery device, for deploying the grommet into the lumen of a patient. The pusher may include a monolithically formed pushrod or wire, cable, or articulating mechanism the advances the grommet from the access device. The pusher may be rigid, semi-rigid, or flexible.

The medical professional advances the access device into the distal lumen and deploys the distal flange of the apparatus, with the proximal flange remaining inside the access device. The distal flange may be deployed by pulling back on the access device, advancing the secondary delivery device, and/or advancing the pusher. After the distal flange is deployed into the lumen, on the distal side of the otomy, the distal flange expands into the deployed configuration.

The medical professional pulls back on the access device into the body cavity from the lumen. The medical professional then pulls back on the access device, pulls back on the secondary delivery device, and/or advances the pusher to deploy the proximal flange on the proximal side of the tissue surrounding the otomy. Upon deploying from the access device, the proximal flange expands into the deployed configuration. As shown in FIG. 2B, the grommet is then in the fully deployed configuration, with the distal flange engaging the distal side of the tissue surrounding the otomy within the lumen, and the proximal flange engaging the proximal side of the tissue surrounding the otomy.

In some embodiments, the medical professional may insert the access device endoscopically into a lumen. From inside the lumen, the medical professional advances the access device through the tissue wall into the body cavity. The distal flange may be deployed by pulling back on the access device, advancing the secondary delivery device, and/or advancing the pusher. After the distal flange is deployed into the body cavity, on the distal side of the otomy, the distal flange expands into the deployed configuration.

The medical professional pulls back on the access device into the lumen from the body cavity. The medical professional then pulls back on the access device, pulls back on the secondary delivery device, and/or advances the pusher to deploy the proximal flange on the proximal side of the tissue surrounding the otomy. Upon deploying from the access device, the proximal flange expands into the deployed configuration. As shown in FIG. 2B, the grommet is then in the fully deployed configuration, with the distal flange engaging the distal side of the tissue surrounding the otomy within the body cavity, and the proximal flange engaging the proximal side of the tissue surrounding the otomy within the lumen.

In the fully deployed configuration, as shown in FIG. 2B, the grommet allows for control of the otomy, reduction tissue dilation/tearing around the otomy, and a channel for immediate fluidic flow through the otomy.

After the grommet is deployed within the otomy, the medical professional may advance an access device through the otomy for further surgical procedures, as shown in FIG. 2C. For example, in the creation of an anastomosis, the medical professional may deploy a magnetic compression anastomosis device or other device into the lumen of the organ/bowel of a patient at a target anastomosis site through the grommet. Additionally or alternatively, the medical professional may deploy a magnetic compression anastomosis device or other device into the lumen prior to deployment of the grommet. FIG. 2D shows a magnetic compression anastomosis device deployed in the lumen behind the grommet. Whether the magnetic compression anastomosis device is deployed before or after the grommet, the diameter of the magnetic compression anastomosis device is generally greater than the diameter of the grommet and therefore is unable to pass through the grommet out of the lumen.

The methods and steps as described above and depicted in FIGS. 2A-2D may be repeated in another organ/bowel/lumen of the patient at another target anastomosis site one or more times. The medical professional then can bring the otomies of the one or more target sites in proximity with each other. The otomies may be manipulated, for example, endoscopically, laparoscopically, robotically, or in open field surgery. The medical professional may manipulate the otomies, for example, using sutures attached to the magnetic compression anastomosis devices, as is shown in FIG. 2E.

After the magnetic compression anastomosis devices are in proximity with each other, they mate due to attractive magnetic forces as shown in FIG. 2F, compressing the tissue therebetween to form an anastomosis. As is shown in FIG. 2G, the channel within the grommets allows for immediate patency and fluidic passage between the lumens before the complete creation of an anastomosis.

The magnetic compression anastomosis devices compress and necrose the tissue therebetween. As the tissue necroses, the grommets may fall away from the target site and pass naturally from the patient, as is shown in FIG. 2H. After complete formation of the anastomosis, the magnetic compression anastomosis devices also fall away from the target site and pass naturally from the patient, as is shown in FIG. 2I, leaving a fully formed anastomosis.

Compression anastomosis devices described herein may include articulating magnetic compression anastomosis devices, which may be self-assembling. For example, the magnetic anastomosis devices of certain embodiments generally comprise magnetic segments that can assume a delivery conformation and a deployed configuration. The delivery configuration is typically linear so that the device can be delivered to a tissue via a laparoscopic “keyhole” incision or with delivery via a natural pathway, e.g., via the esophagus, with an endoscope or similar device. Additionally, the delivery conformation is typically somewhat flexible so that the device can be guided through various curves in the body. Once the device is delivered, the device will assume a deployed configuration of the desired shape and size by converting from the delivery configuration to the deployed configuration automatically. The self-conversion from the delivery configuration to the deployment configuration is directed by coupling structures that cause the magnetic segments to move in the desired way without intervention.

In general, a magnetic anastomosis procedure involves placing a first and a second magnetic structure adjacent to first and second portions of tissues, respectively, thus causing the tissues to come together. Once the two devices are brought into proximity, the magnetic structures mate and bring the tissues together. With time, an anastomosis of the size and shape of the devices will form and the devices will fall away from the tissue. In particular, the tissues circumscribed by the devices will be allowed to necrose and degrade, providing an opening between the tissues.

FIG. 3 depicts a side-view of the exemplary grommet otomy control apparatus of FIG. 1. An exemplary grommet has a proximal flange and a distal flange connected by a channel therebetween. The diameter of the channel is less than the diameter of the flanges. As is shown in FIG. 3, the grommet is deployed across the otomy, with the one flange engaging the intralumenal side of the tissue and the other flange engaging the extraluminal side of the tissue. The inner diameter channel allows for immediate patency and fluidic passage through the otomy. Additionally, the passage allows for deployment of a compression anastomosis device, as shown in FIG. 3, within the lumen on the distal side of the otomy.

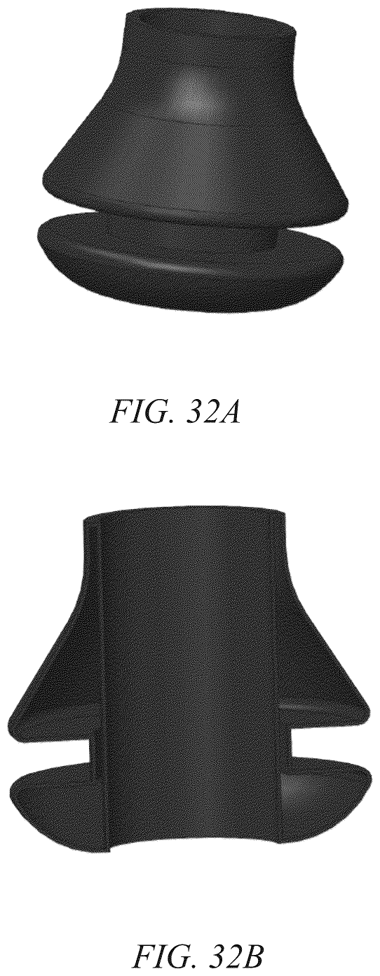

In various embodiments, the otomy control device includes an inner framework and an external covering encompassing some or all of the inner framework. Generally speaking, the inner framework and/or the external covering may include flexible portions (e.g., portions associated with the flanges) that allow for the otomy control device to be compressed within the delivery device but also for the otomy control device flanges to automatically expand or unfold when deployed from the delivery device. Thus, for example, the inner framework may include or be formed from shape-memory material (e.g., a nitinol or other shape memory material such as a spring material), and the external covering may be made of any appropriate material (e.g., plastic, rubber, urethane, polymer, etc.) and may be formed using any appropriate technique (e.g., lamination, injection molding, etc.). The inner framework and/or external covering made include a biocompatible material. Various alternative otomy control devices are described herein, and it should be understood that some embodiments depict configurations that may be used alone and/or with an external covering (e.g., some embodiments depict configurations that may be the inner framework on or over which an external covering may be formed). It also should be understood that some embodiments can be formed without an inner framework. For example, certain devices (e.g., as shown in FIG. 1 and FIGS. 32A/B) can be formed as a unitary device (e.g., injection molded) with flanges that are sufficiently flexible for storage in the delivery device and expansion upon deployment.

In various embodiments, magnets may be incorporated into the grommet (e.g., in one or both of the flanges and/or about the center channel section), combining the grommet and magnetic compression anastomosis device into a single apparatus. The magnet or magnets may be incorporated into the distal and/or proximal flange, or the entire grommet. This would allow for the grommet to control the otomy, while also acting as the magnetic compression anastomosis device.

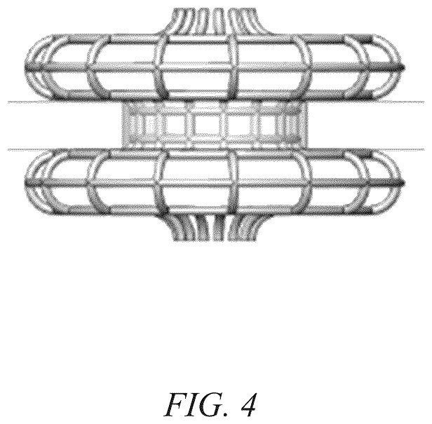

FIG. 4 depicts an exemplary embodiment of a grommet-style otomy control apparatus. The grommet depicted in FIG. 4 comprises a semi-rigid or rigid conduit that acts as an open channel to facilitate deployment and alignment of surgical tools as well as fluidic passage through the otomy. The grommet may be constructed from nitinol, stainless steel, or other bio-compatible materials.

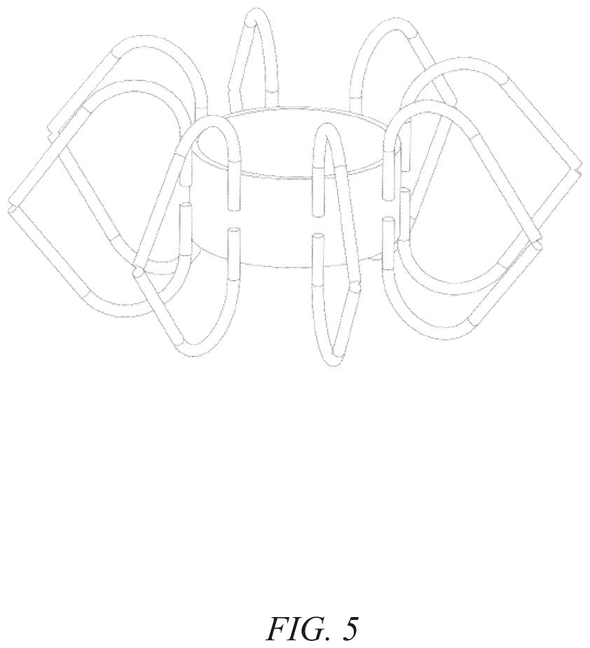

FIG. 5 depicts a nitinol tubing or wire hybrid grommet. Nitinol wires or tubes may be attached to a central tube section to clamp a periphery of tissue surrounding an otomy from both sides. The central tube section allows for passage of fluid and surgical tools through the otomy while preserving the shape and size and reducing tearing of the tissue surrounding the otomy. The central tube section can be made of any appropriate material, e.g., nitinol, stainless steel, etc. In order to deliver this device into a patient, the device would be stored inside a delivery device such as an endoscope, laparoscope, catheter, etc. with the top set of nitinol wires/tubes extended upward and with the bottom set of nitinol wires/tubes extended downward. Note that the embodiments shown in FIGS. 5-6B, 13A-19B, 21A-25B may be delivered into the patient in this manner.

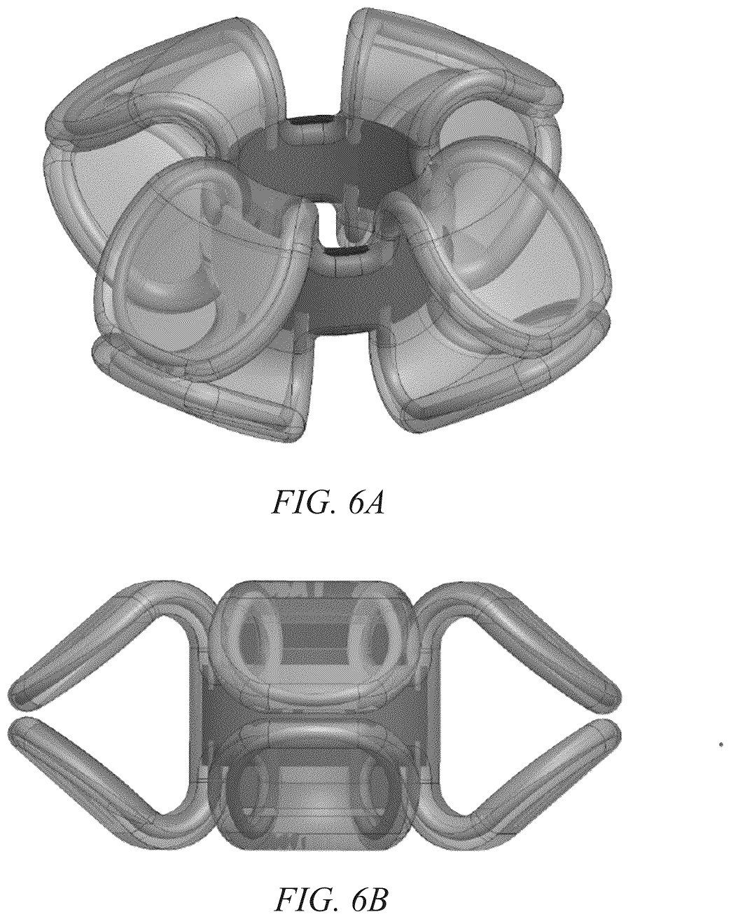

FIGS. 6A-6B depict another exemplary nitinol tubing or wire hybrid grommet. The grommet may comprise two or more bent wire loops attached to a tube section. FIGS. 6A-6B depict a wire hybrid grommet comprising four bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy. The embodiment shown in FIGS. 6A-6B depict an inner framework covered by an external covering, as discussed above.

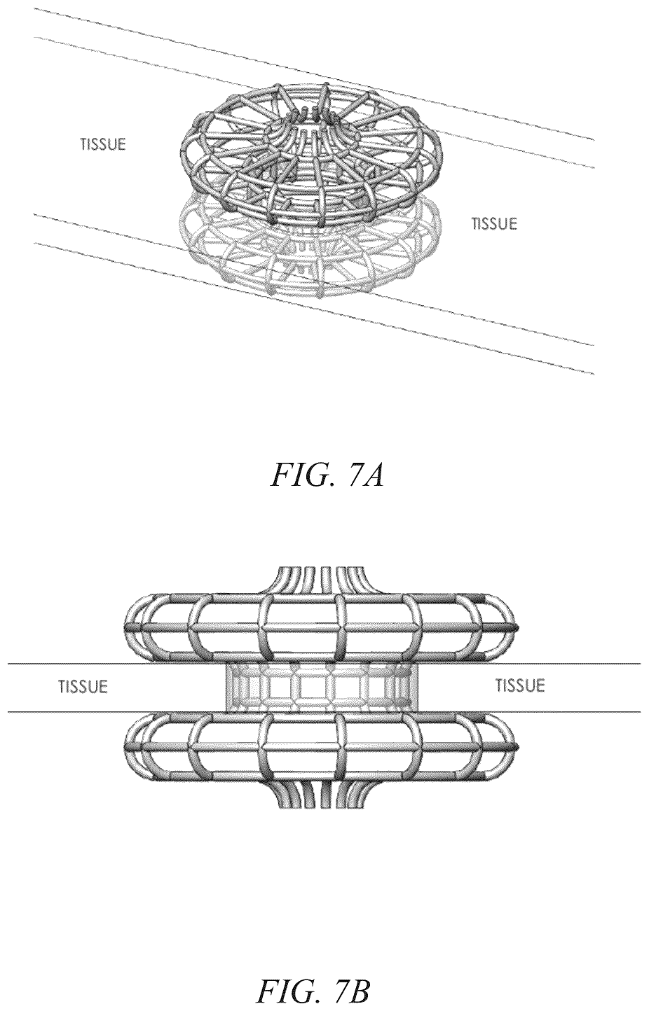

FIGS. 7A-7B depict nitinol wire stent array grommets. The nitinol wire stent array clamps the tissue surrounding an otomy site, securing the otomy and reducing tissue deformation around the otomy site. As is depicted in FIG. 7B, the flanges of the nitinol wire stent array engage the distal and proximal sides of the tissue respectively, while the tube section passes through the otomy, allowing for immediate patency and fluid passage before complete formation of the permanent anastomosis. The tube section also maintains the diameter of the otomy as the flanges reduce tearing and dilation of the tissue surrounding the otomy during a surgical procedure.

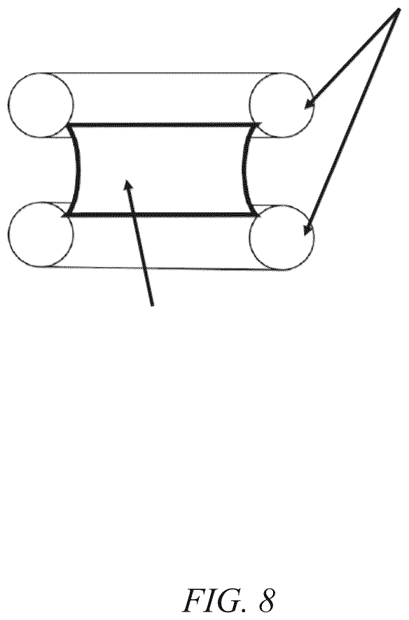

FIG. 8 depicts an exemplary embodiment of an inflatable grommet. An inflatable grommet is deflated in a delivery configuration within an access device. After deployment into the distal lumen of an otomy, the distal flange of the grommet is inflated to a deployed diameter. The proximal flange is then deployed on the proximal side of the otomy and inflated to a deployed diameter. Once inflated into the deployed configuration, the inflatable grommet secures the otomy and reduces tearing of tissue around the otomy site.

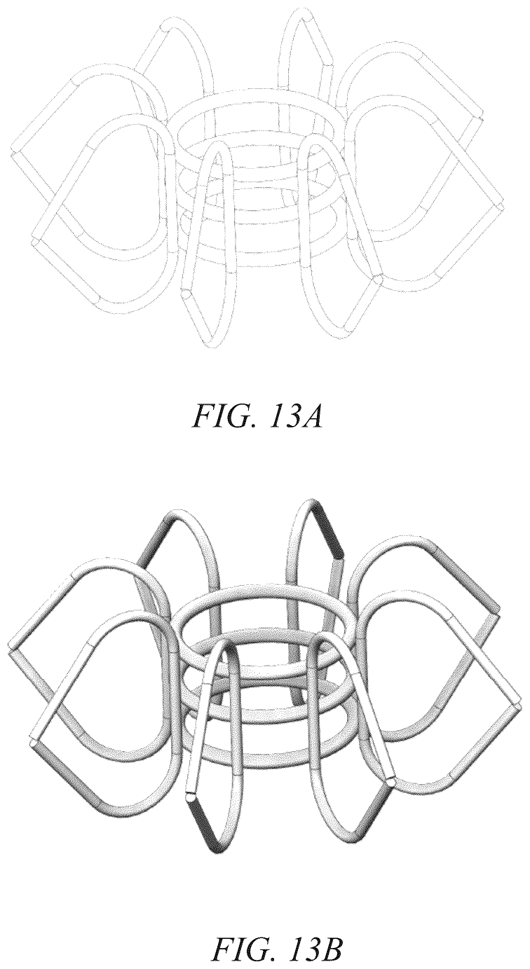

FIGS. 13A-13B depict perspective views of an exemplary nitinol tubing/wire hybrid grommet for securing an acute otomy. This device is similar to the device shown in FIG. 5 but includes a central open ring section rather than a central tube section to which the nitinol wires or tubes are attached, forming a nitinol wire or tubing array that clamps a periphery of tissue surrounding an otomy site from both sides. The array has bent tube or wire sections to clamp tissue, while the hollow center section allows for immediate patency through the otomy and fluid passage before complete formation of the permanent anastomosis. The center section also maintains the diameter of the otomy as the nitinol wires/tubing reduces tearing and dilation of the tissue surrounding the otomy during a surgical procedure. The central open ring section can be made of any appropriate material, e.g., nitinol, stainless steel, etc. This device can be delivered by storing the device in a delivery device as described above with reference to FIG. 5.

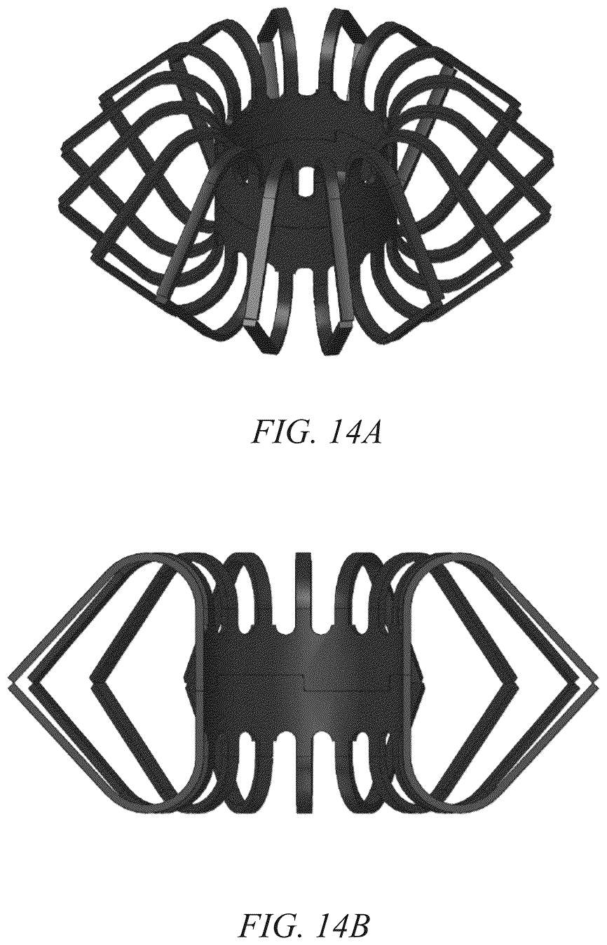

FIGS. 14A-14B depict a two-piece nitinol tubing/wire hybrid grommet. Nitinol wires may be attached to a center section to clamp a periphery of tissue surrounding an otomy. The two halves of the nitinol tubing/wire array grommet are stored in a delivery device in a delivery configuration. The medical professional advances the delivery device through a created otomy, and pulls back on the delivery device to deploy the distal half of the nitinol tubing/wire array grommet into a deployed configuration. The medical professional may then pull the delivery device further back to deploy the proximal half of the grommet into a deployed configuration. The outer arms of the two halves clamp the periphery tissue of the created otomy, while the center portion allows for passage of fluid and surgical tools through the otomy. This preserves the shape and size and reducing tearing of the tissue surrounding the otomy. The two halves of the array grommet may be held together mechanically, magnetically, and/or by other mechanism. As depicted in FIGS. 14A-14B, the two halves have corresponding mating elements to better ensure that the two halves join in a desired configuration (which in this embodiment aligns corresponding arms of the two halves).

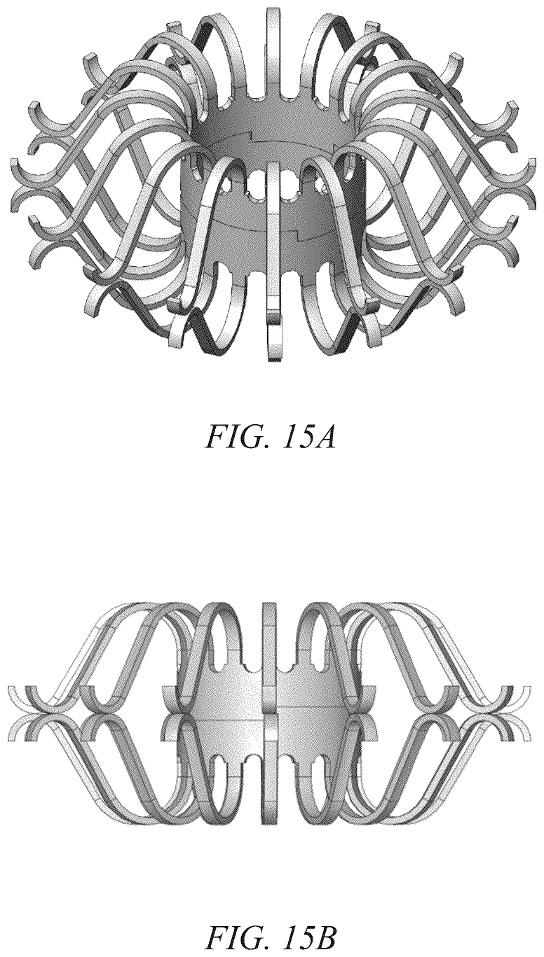

FIGS. 15A-15B depict an exemplary two-piece nitinol tubing/wire grommet. Nitinol wires may be attached to a center section to clamp a periphery of tissue surrounding an otomy. The center portion allows for passage of fluid and surgical tools through the otomy while preserving the shape and size and reducing tearing of the tissue surrounding the otomy. The two halves of the array grommet may be held together mechanically, magnetically, and/or by other mechanism. As depicted in FIGS. 15A-15B, the two halves have corresponding mating elements to better ensure that the two halves join in a desired configuration (which in this embodiment aligns corresponding arms of the two halves).

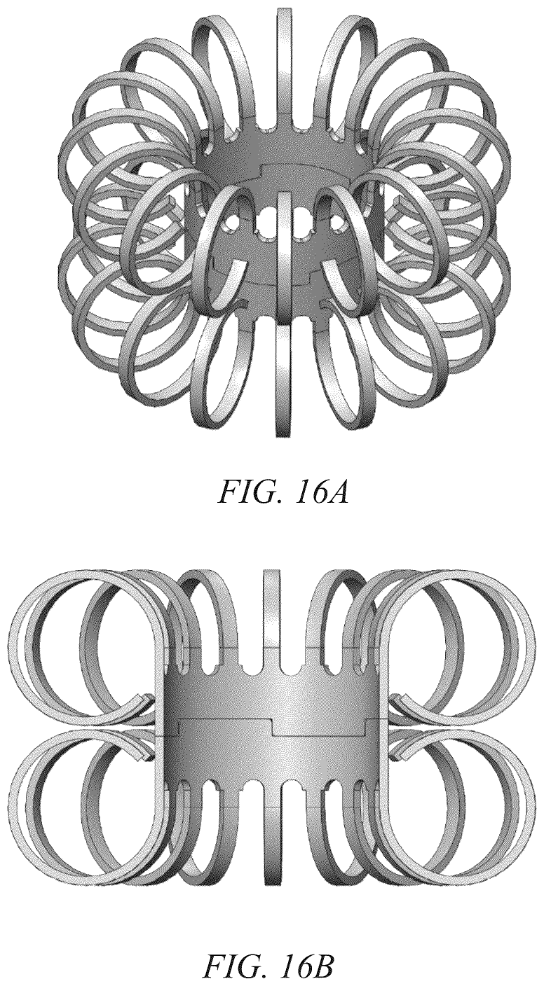

FIGS. 16A-16B depict perspective views of an exemplary two-piece nitinol tubing/wire array grommet. Nitinol tubing/wire arms may be attached to the circumference of a center section of the grommet. The arms clasp the tissue surrounding a created otomy while the center section allows for immediate passage of fluids and other materials such as surgical tools through the otomy before the creation of a permanent anastomosis. This secures the tissue surrounding the otomy site, reducing tearing, dilation, or locomotion of the otomy site.

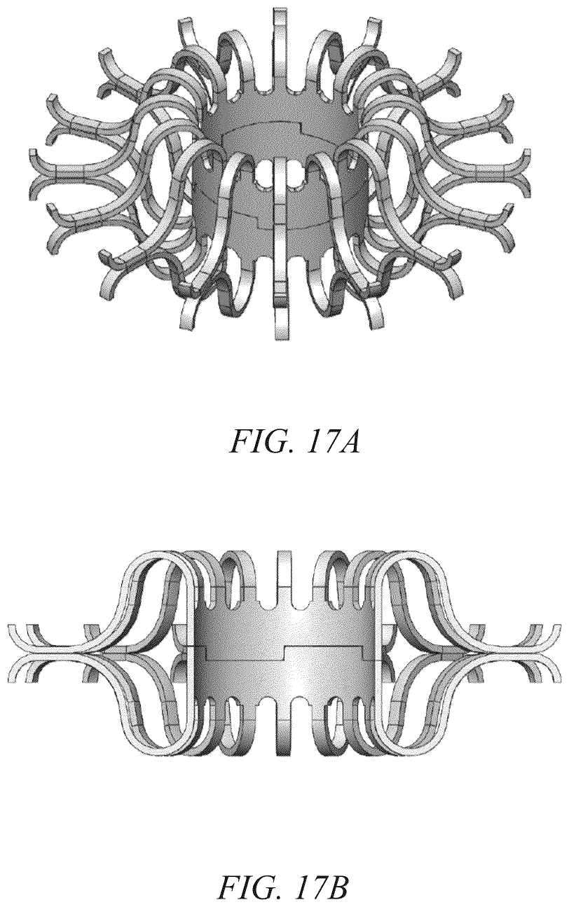

FIGS. 17A-17B depict another embodiment of a two-piece nitinol tubing/wire array grommet. Nitinol tubing/wire arms may be attached to the circumference of a center section of the grommet. The arms clasp the tissue surrounding a created otomy while the center section allows for immediate passage of fluids and other materials such as surgical tools through the otomy before the creation of a permanent anastomosis. This secures the tissue surrounding the otomy site, reducing tearing, dilation, or locomotion of the otomy site.

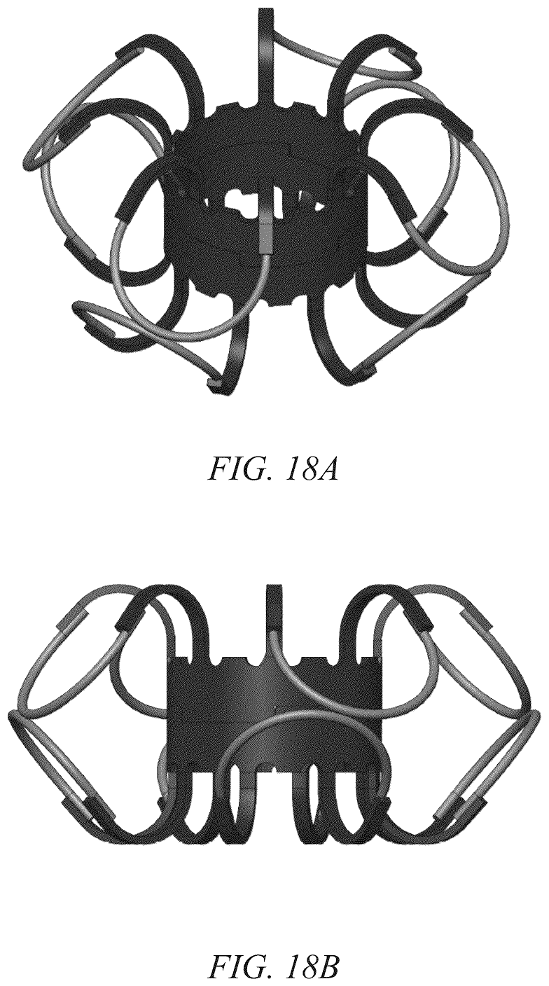

FIGS. 18A-18B depict perspective views of another embodiment of a two-piece nitinol tubing/wire array grommet. The grommet may comprise two or more bent wire loops attached to a tube section. FIGS. 18A-18B depict a wire hybrid grommet comprising eight bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy.

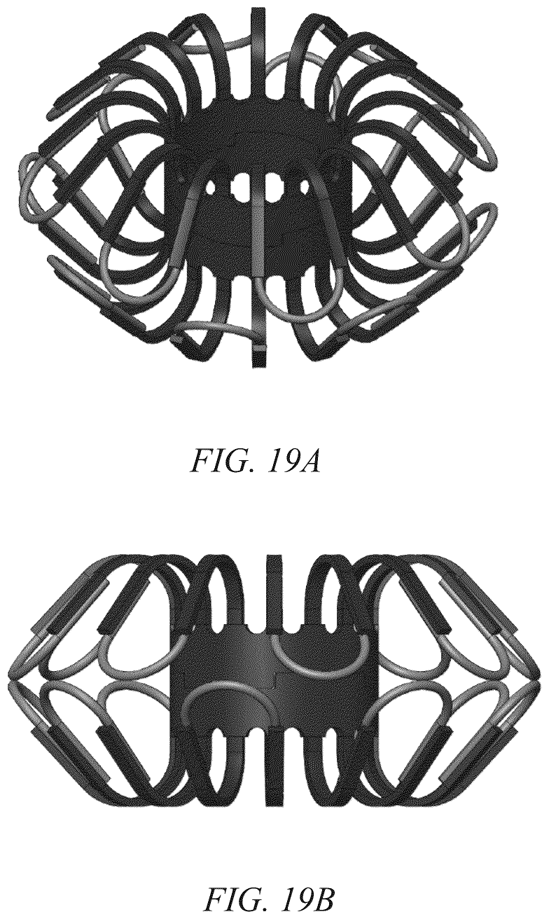

FIGS. 19A-19B depict perspective views of another embodiment of a two-piece nitinol tubing/wire array grommet. The grommet may comprise two or more bent wire loops attached to a tube section. FIGS. 19A-19B depict a wire hybrid grommet comprising sixteen bent wire loops. The wire loops may be constructed from nitinol, stainless steel, or other bio-compatible materials. The bent wire loops clamp the tissue surrounding the otomy, securing the size and shape of the otomy and reducing tissue tearing. The tube section allows for the passage of fluid or surgical tools through the otomy.

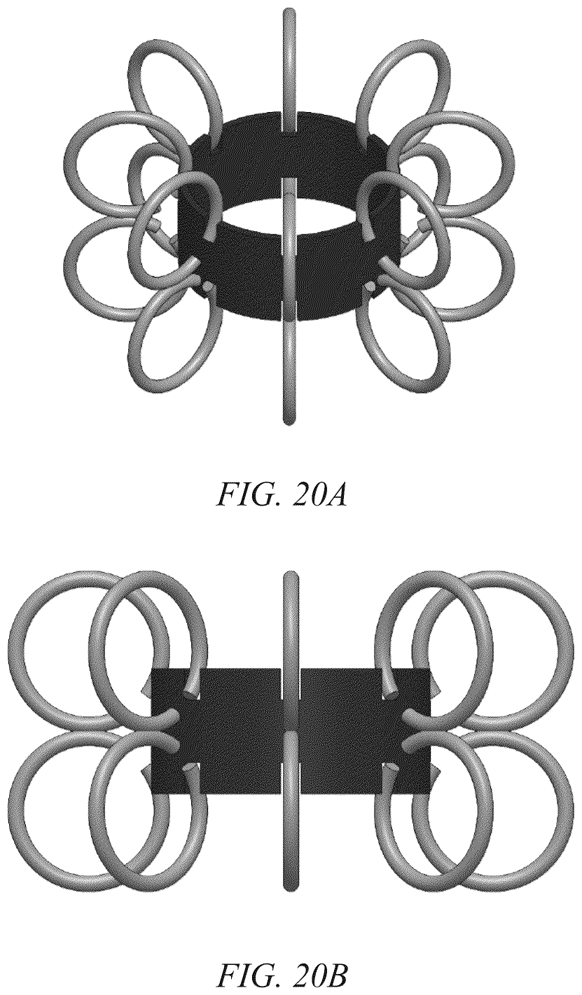

FIGS. 20A-20B depict perspective views of another embodiment of a nitinol tubing/wire array grommet. The grommet of FIGS. 20A-20B may comprise two or more bent wire loops. FIGS. 20A-20B depict a grommet comprising sixteen wire loops, eight on the distal half of the grommet, and eight on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

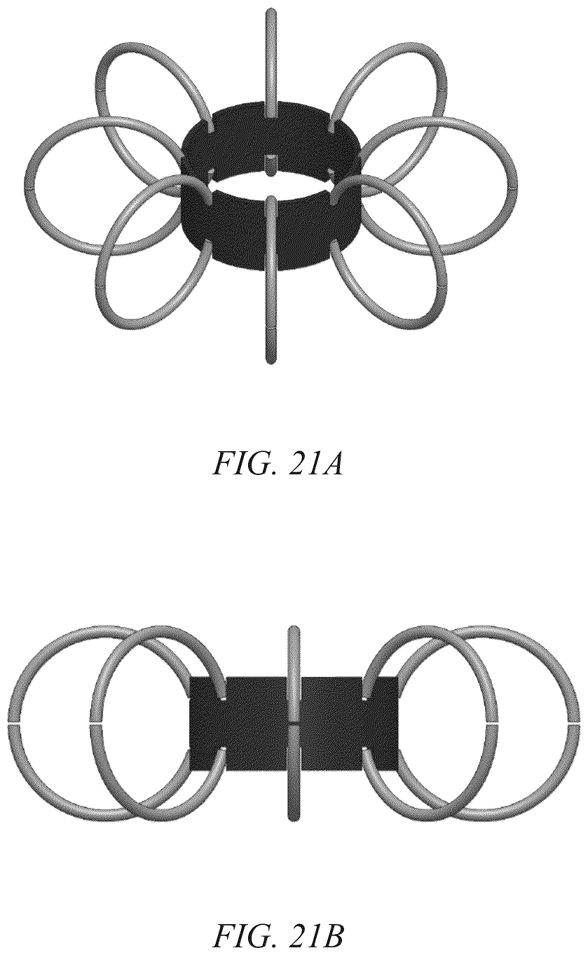

FIGS. 21A-21B depict an embodiment of a nitinol tubing/wire array grommet comprising wire half loops. The grommet of FIGS. 21A-21B comprise sixteen wire half-loops, with eight half loops on the proximal half of the device, eight half loops on the distal half of the device, and a hollow center section. The grommet device is stored in a delivery device in a delivery configuration. The medical professional advances the delivery device through a created otomy. Once on the distal side of the otomy, the medical professional pulls back on the delivery device to deploy the distal half of the grommet on the distal side of the tissue surrounding the otomy into a deployed configuration. The medical professional may pull further back on the delivery device through to the proximal side of the otomy to deploy the proximal half of the grommet device on the proximal side of the tissue surrounding the otomy into a deployed configuration. Once in the deployed configuration, the half loops of the distal half come into proximity with the half loops of the proximal half of the device to clasp the tissue surrounding the otomy. This secures the size and shape of the otomy and reduces tissue tearing. The center section of the grommet allows for the passage of fluid or surgical tools through the otomy.

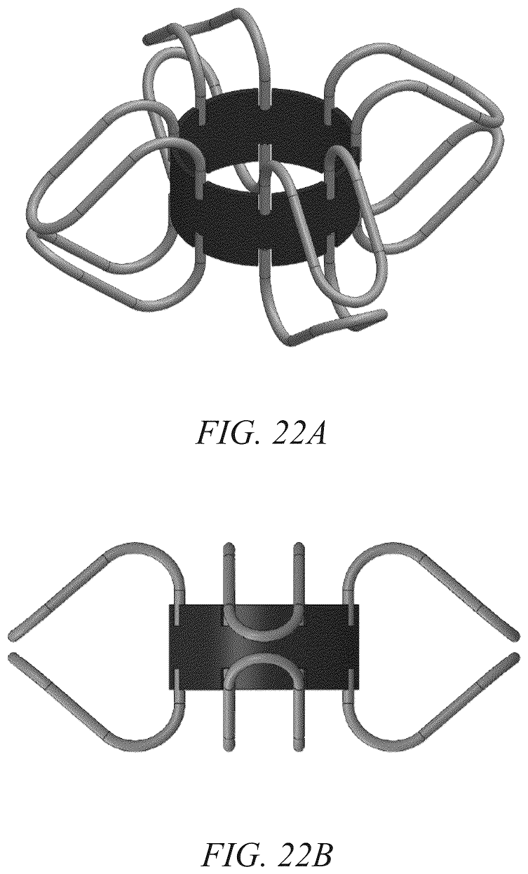

FIGS. 22A-22B depict perspective views of another embodiment of a nitinol tubing/wire array grommet. The grommet of FIGS. 22A-22B may comprise two or more bent wire loops. FIGS. 22A-22B depict a grommet comprising eight wire loops, four on the distal half of the grommet, and four on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

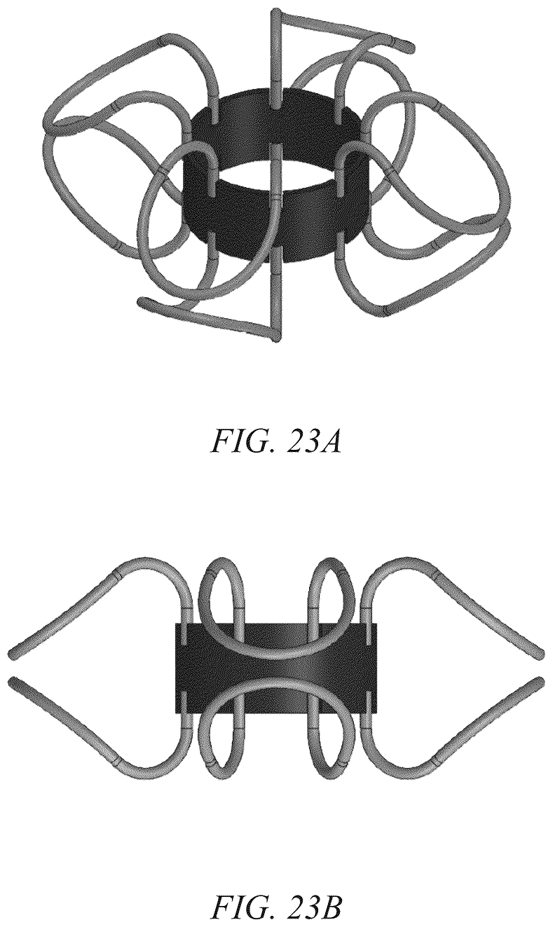

FIGS. 23A-23B depict perspective views of another embodiment of a nitinol tubing/wire array grommet. The grommet of FIGS. 23A-23B may comprise two or more bent wire loops. FIGS. 23A-23B depict a grommet comprising eight wire loops, four on the distal half of the grommet, and four on the proximal half. The wire loops may be constructed from nitinol, stainless steel, or other bio-compatible materials. The bent wire loops clamp the tissue surrounding the otomy, with the circumference of the loops perpendicular to the tissue surface. This secures the size and shape of the otomy and reduces tissue tearing. The center section of the grommet allows for the passage of fluid or surgical tools through the otomy.

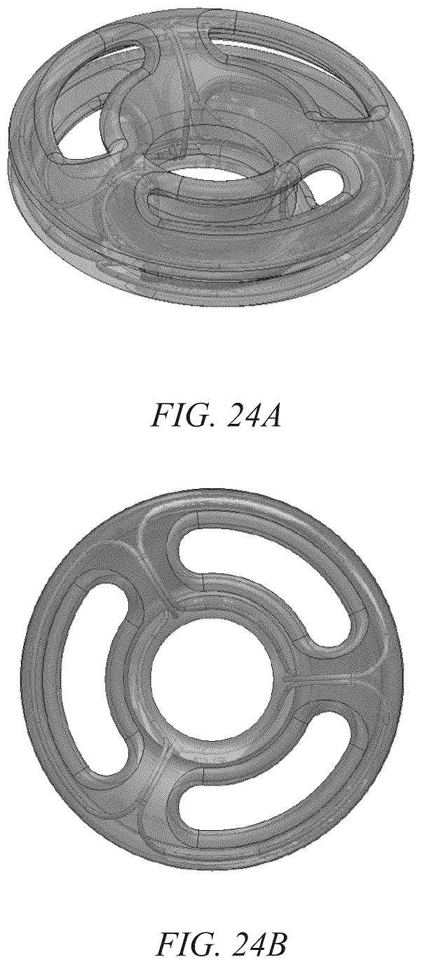

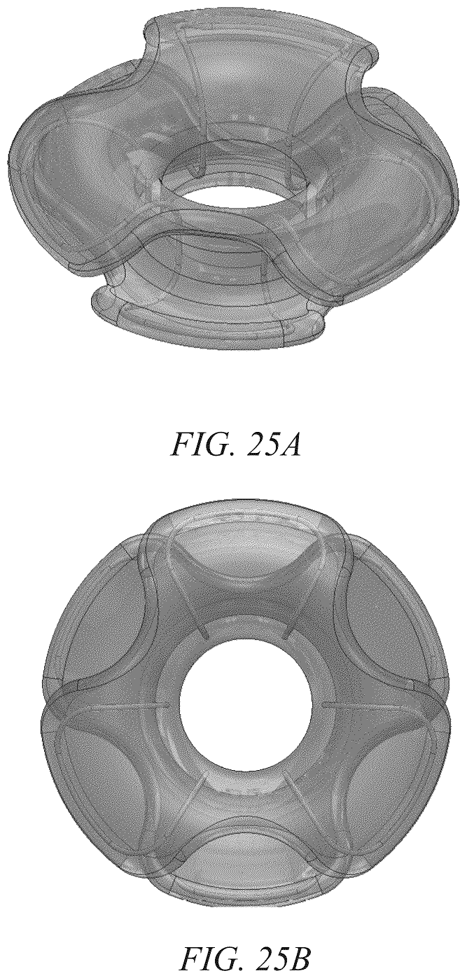

FIGS. 24A-24B and FIGS. 25A-25B depict an embodiment of a nitinol tubing/wire hybrid grommet. The grommet may comprise two or more wire loops enveloped in a bio-compatible material, creating a distal flange and a proximal flange. The flanges may be continuous as shown in FIGS. 25A-25B or may comprise cutaway sections as shown in FIGS. 24A-24B. A hollow center section connecting the two flanges may be present. The grommet device is stored in a delivery device in a delivery configuration. The medical professional advances the delivery device through a created otomy. Once on the distal side of the otomy, the medical professional pulls back on the delivery device to deploy the distal flange of the grommet into the distal side of the tissue surrounding the otomy into a deployed configuration. The medical professional may pull further back on the delivery device through to the proximal side of the otomy to deploy the proximal flange of the grommet device in the proximal side of the tissue surrounding the otomy into a deployed configuration. Once in the deployed configuration, the flanges clasp the tissue surrounding the otomy. This secures the size and shape of the otomy and reduces tissue tearing. The center section of the grommet allows for the passage of fluid or surgical tools through the otomy. The embodiment shown in FIGS. 24A-24B and FIGS. 25A-25B depict an inner framework covered by an external covering, as discussed above.

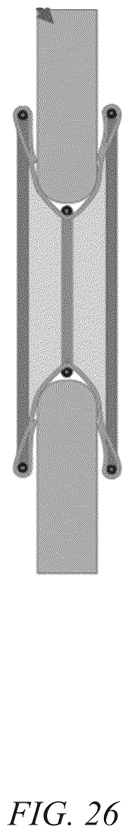

FIG. 26 depicts a triple sleeved hoop device for securing tissue surrounding a created otomy. The otomy sleeve comprises a small, central hoop with two larger hoops parallel to the central hoop, one on each side. The small hoop provides a work path/conduit centered on the otomy for fluid and/or surgical tools to pass through the otomy. The larger hoops tension a sleeve and prevent the device from being pulled out of the otomy. The sleeved hoop is stored in a delivery device in a delivery configuration. The medical professional advances the delivery device through a created otomy. Once on the distal side of the otomy, the medical professional pulls back on the delivery device to deploy the distal larger hoop of the triple sleeved hoop into the distal side of the tissue surrounding the otomy into a deployed configuration. The medical professional may pull further back on the delivery device through to the proximal side of the otomy to deploy the proximal larger hoop of the triple sleeved hoop in the proximal side of the tissue surrounding the otomy into a deployed configuration. Once in the deployed configuration, the larger hoops clasp the tissue surrounding the otomy. This secures the size and shape of the otomy and reduces tissue tearing. The central hoop allows for the passage of fluid or surgical tools through the otomy.

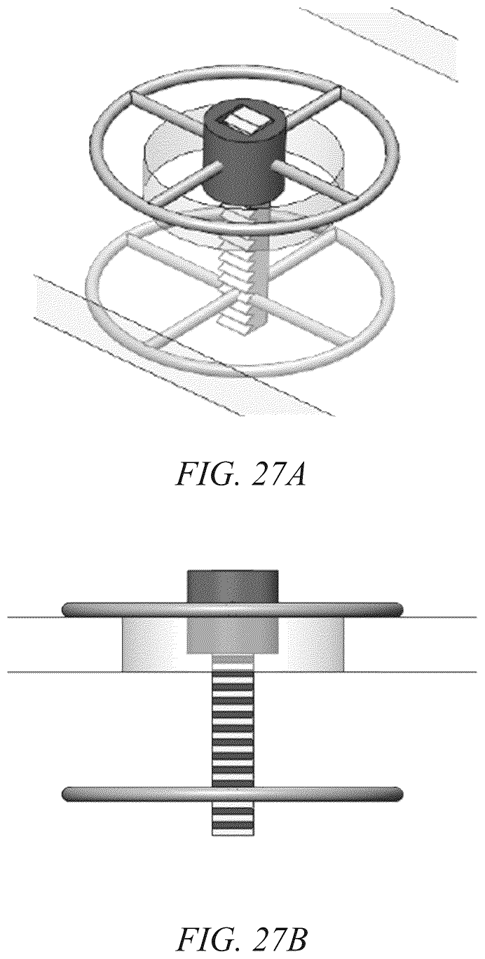

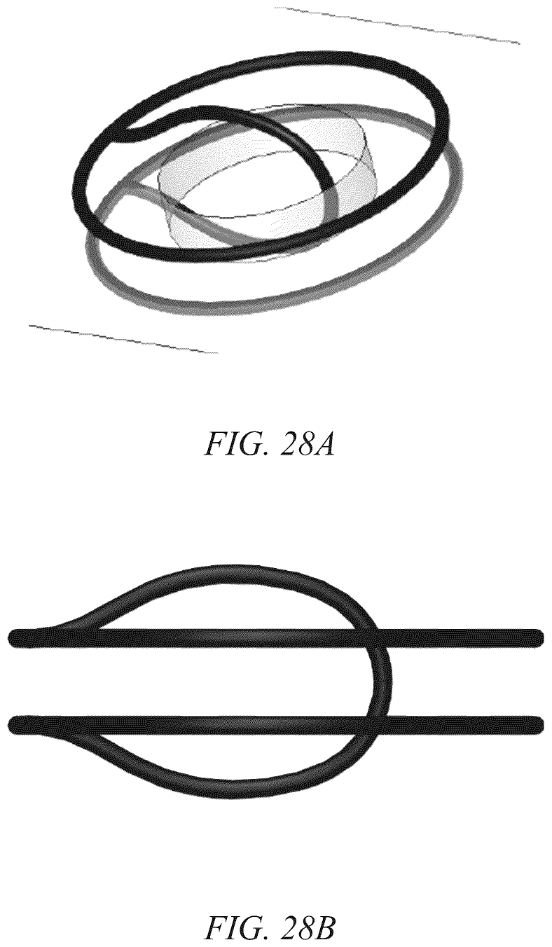

FIGS. 27A-27B depict a ratcheting otomy securement apparatus. An exemplary embodiment comprises a distal ratcheting member and a proximal ratcheting member connected by a central ratchet. The ratcheting securement apparatus is stored in a delivery device in a delivery configuration. The medical professional advances the delivery device through a created otomy. Once on the distal side of the otomy, the medical professional pulls back on the delivery device to deploy the distal ratcheting member on the distal side of the tissue surrounding the otomy into a deployed configuration. The medical professional may pull further back on the delivery device through to the proximal side of the otomy to deploy the proximal ratcheting member on the proximal side of the tissue surrounding the otomy into a deployed configuration. Once in the deployed configuration, the medical professional may ratchet the two ratcheting members toward each other. The central ratchet only allows for the ratcheting members to move into proximity, not further apart. Once in proximity to one another, the ratcheting members clamp the tissue surrounding the otomy, securing the otomy and reducing tissue damage. It should be noted that the otomy securement apparatus may include alternative mechanisms for drawing the two members toward each other and/or preventing the two members from moving apart from one another after being brought into proximity, e.g., a screw mechanism, a locking mechanism, etc. For convenience, the ratchet mechanism or alternative mechanisms are referred to herein as locking mechanisms. Generally speaking, any of the two-piece otomy control devices with distal and proximal flanges described herein can be configured to include a locking mechanism to interlock with another otomy control device.

FIGS. 28A-28B depict a circular otomy securement device. The circular securement device may be made from nitinol, stainless steel, or other biocompatible materials. As is shown in FIG. 28A, the securement device may comprise two circular hoops parallel to each other and connected by one or more central supports. The circular securement device is stored in a delivery device in a delivery configuration. The medical professional advances the delivery device through a created otomy. Once on the distal side of the otomy, the medical professional pulls back on the delivery device to deploy the distal hoop into the distal side of the tissue surrounding the otomy into a deployed configuration. The medical professional may pull further back on the delivery device through to the proximal side of the otomy to deploy the proximal hoop in the proximal side of the tissue surrounding the otomy into a deployed configuration. Once in the deployed configuration, the hoops, connected by a central support, clasp the otomy. This secures the size and shape of the otomy and reduces tissue tearing. The central opening of the hoops allows for immediate passage of fluid and other materials through the otomy before permanent anastomosis formation.

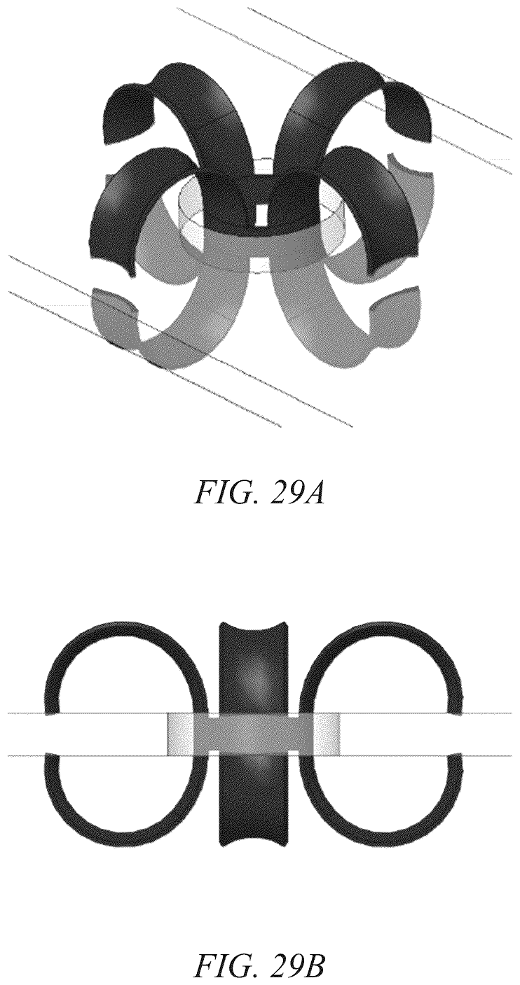

FIGS. 29A-29B depict a stent-style nitinol array. In an embodiment of the device, split nitinol tubing creates arms that may clamp tissue surrounding an otomy site. The arms are positioned around the circumference of a hollow central portion of the device. The stent-style nitinol array is stored in a delivery device in a delivery configuration. The medical professional advances the delivery device through a created otomy. Once on the distal side of the otomy, the medical professional pulls back on the delivery device to deploy distal arms of the device into the distal side of the tissue surrounding the otomy into a deployed configuration. The medical professional may pull further back on the delivery device through to the proximal side of the otomy to deploy proximal arms of the device in the proximal side of the tissue surrounding the otomy into a deployed configuration. Once in the deployed configuration, the arms clasp the tissue surrounding the otomy. This secures the size and shape of the otomy and reduces tissue tearing. The central section of the device allows for the passage of fluid or surgical tools through the otomy.

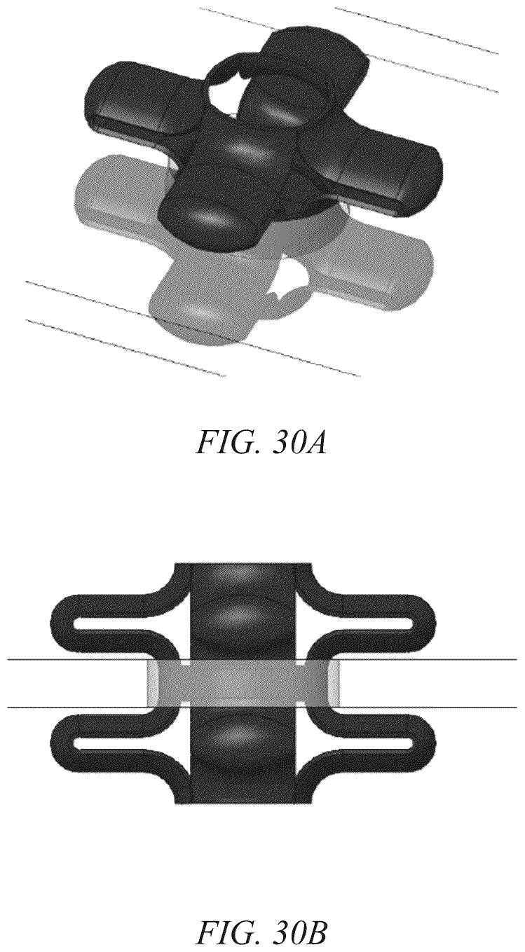

FIGS. 30A-30B depict an alternative embodiment of a stent-style nitinol array securement device. The embodiment may comprise nitinol tubing that deforms upon deployment to clamp the tissue surrounding an otomy. The apparatus may comprise two or more nitinol tubes around the circumference of a hollow center region. The stent-style nitinol array is stored in a delivery device in a delivery configuration. The medical professional advances the delivery device through a created otomy. Once on the distal side of the otomy, the medical professional pulls back on the delivery device to deploy the distal nitinol tubes into the distal side of the tissue where they deform into a deployed configuration. The medical professional may pull further back on the delivery device through to the proximal side of the otomy to deploy the proximal nitinol tubes into the proximal side of the tissue where they deform into a deployed configuration. Once in the deployed configuration, the tubes clasp the tissue surrounding the otomy. This secures the size and shape of the otomy and reduces tissue tearing. The center section allows for immediate passage of fluid and/or surgical tools through the otomy.

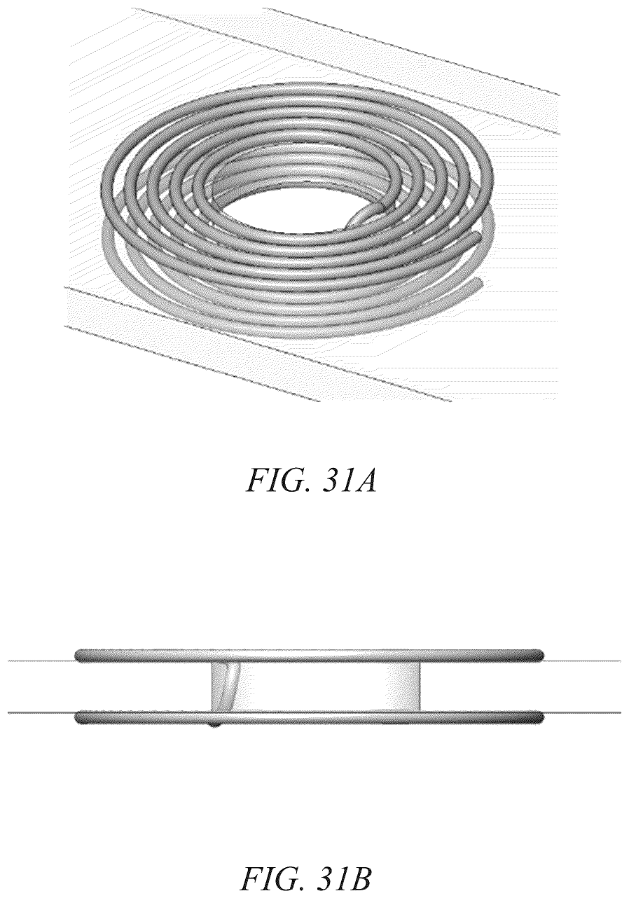

FIGS. 31A-31B depict a spiral style stent-style nitinol array securement device. A single length of nitinol, stainless steel, or other biocompatible material is coiled into two parallel, or substantially parallel, spiral arrays connected by a central connecting member. The stent-style nitinol array is stored in a delivery device in a delivery configuration. The medical professional advances the delivery device through a created otomy. Once on the distal side of the otomy, the medical professional pulls back on the delivery device to deploy the distal spiral wire array into the distal side into a deployed configuration. The medical professional may pull further back on the delivery device through to the proximal side of the otomy to deploy the proximal spiral style array into the proximal side of the tissue into a deployed configuration. Once in the deployed configuration, the arrays clasp the tissue surrounding the otomy.

FIGS. 32A-32B depict an embodiment of an inflatable grommet for securing the tissue surrounding an otomy site. The grommet may comprise a distal flange and a proximal flange connected by a hollow central section. The inflatable grommet may be stored in a delivery device in a deflated, delivery configuration. The medical professional advances the delivery device through a created otomy. Once on the distal side of the otomy, the medical professional pulls back on the delivery device to deploy the distal flange of the inflatable grommet into the distal side of the otomy. The medical professional may pull further back on the delivery device through to the proximal side of the otomy to deploy the proximal flange of the inflatable into the proximal side of the tissue. The grommet may then be inflated into a deployed configuration. Once inflated, the flanges clamp the tissue surrounding the otomy, and hollow central portion allows for immediate passage of fluid and other materials through the otomy.

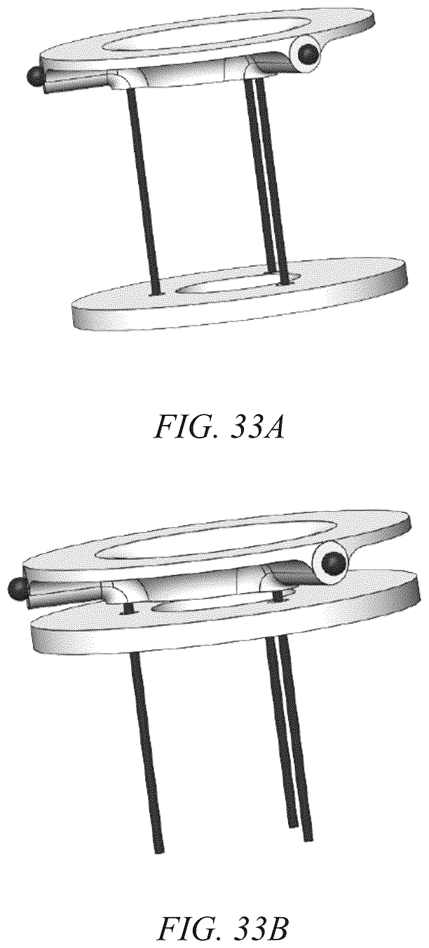

FIGS. 33A-33B depict an embodiment of a two-piece suture clamp. The clamp may comprise two washers made of stainless steel or another biocompatible material aligned parallel or substantially parallel to each other. The washers may be connected by one or more connecting members that are perpendicular to the washers, but parallel to other connecting members. The distal washer is attached to the connecting members. The washers may translate along the connecting members towards or away from the other washer. The medical professional positions a distal washer on the distal side of the created otomy with the connecting members positioned through the otomy. The medical professional then slides the proximal washer onto the connecting members, and translates the proximal washer towards the distal washer. The mated washers clamp tissue surrounding the otomy site, securing the tissue and preserving the otomy. The central opening of the washers allows for immediate passage of fluid and other material through the otomy. Generally speaking, any of the two-piece otomy control devices with distal and proximal flanges described herein can be configured to include one or more connecting members to assist with mating with another otomy control device.

In an alternative embodiment, the proximal washer is attached to the connecting members and the distal washer translates along the connecting members towards the proximal washer.

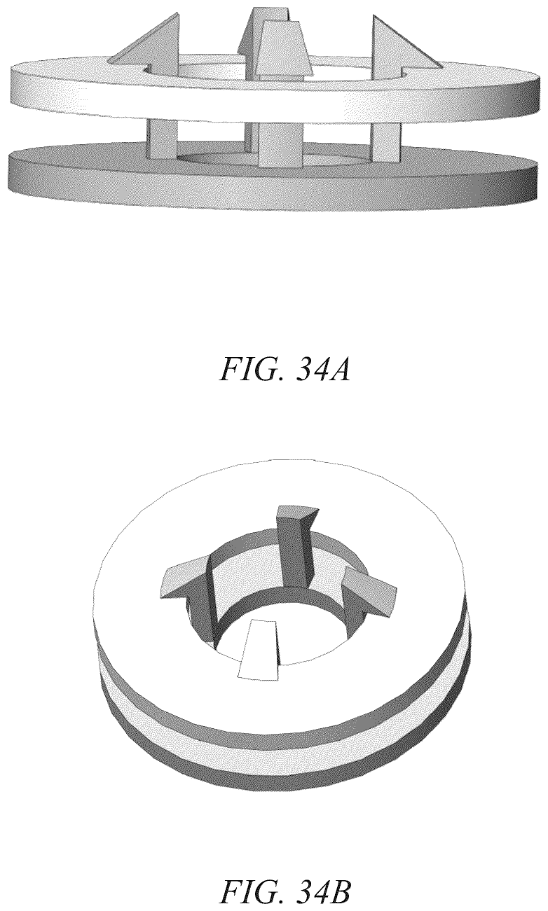

FIGS. 34A-34B depict a snap lock otomy clip, which can be considered another type of locking mechanism as discussed above. The otomy clip may comprise a distal washer and a proximal washer. The distal washer and/or the proximal washer may have snaps configured to mater with the inner circumference of the opposing washer. The medical professional positions the distal washer on the distal side of the otomy and the proximal washer on the proximal side of the otomy. The medical professional then brings the washers into proximity with each other and slides the snaps into the inner circumference of the opposing washer, attaching the washers across the otomy. This secures the tissue surrounding the otomy site and reduces trauma to the tissue while preserving the otomy. Generally speaking, any of the two-piece otomy control devices with distal and proximal flanges described herein can be configured to include a snap lock mechanism to interlock with another otomy control device.

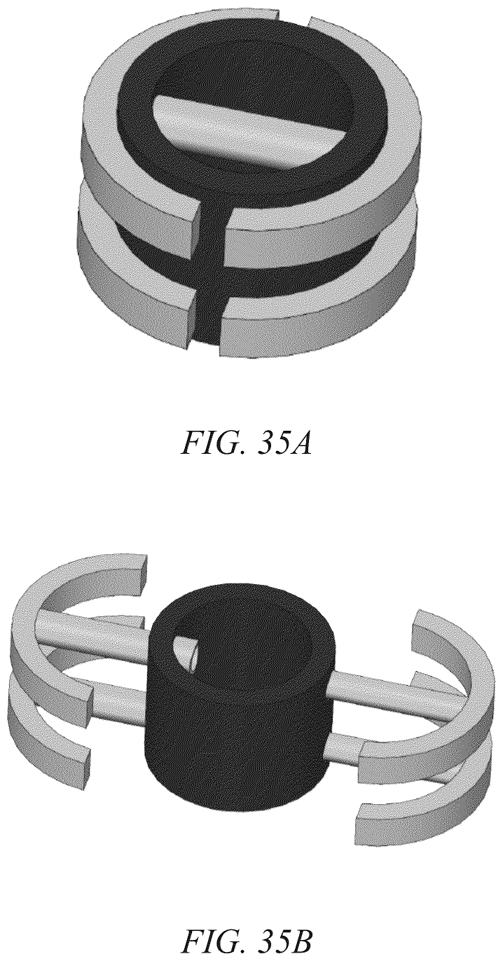

FIGS. 35A-35B depict an embodiment of a sliding arm otomy clip. The otomy clip comprises a hollow central support tube and four or more sliding arms, with two or more being proximal arms and two or more being distal arms. As shown in FIG. 35A, the sliding arms are in a delivery configuration for storage in a delivery device. The arms are curved to compliment the outer circumference of the hollow central support tube. The medical professional advances the delivery device into the otomy so that the distal arms are positioned on the distal side of the otomy and the proximal arms are positioned on the proximal side of the otomy. The medical professional may then deploy the arms of the sliding arm otomy clip into the deployed configurations, as shown in FIG. 35B. The sliding arms protrude from the hollow central support tube and clamp the tissue surrounding the otomy. Note that the arms may deploy in any order.

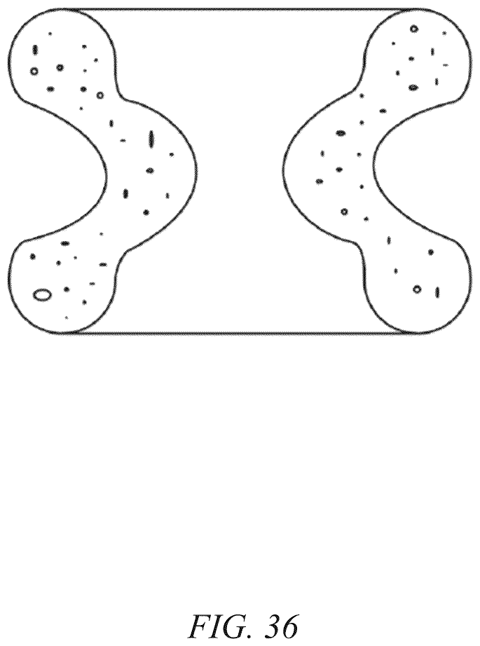

FIG. 36 depicts an embodiment of a foam grommet for securing tissue surrounding an otomy. The foam grommet is stored in a delivery device in a compressed delivery configuration. The medical professional advances the delivery device through the otomy to the distal side of the tissue. The medical professional then pulls back on the delivery device, deploying the distal end of the grommet into the distal lumen. The distal end of the grommet expands into a deployed configuration. The medical professional then pulls back on the delivery device, deploying the proximal end of the grommet into the proximal lumen. The proximal end of the grommet expands into a deployed configuration, as pictured in FIG. 36. The expanded foam grommet comprises a distal flange and a proximal flange, that together clamp and secure the tissue surrounding an otomy.

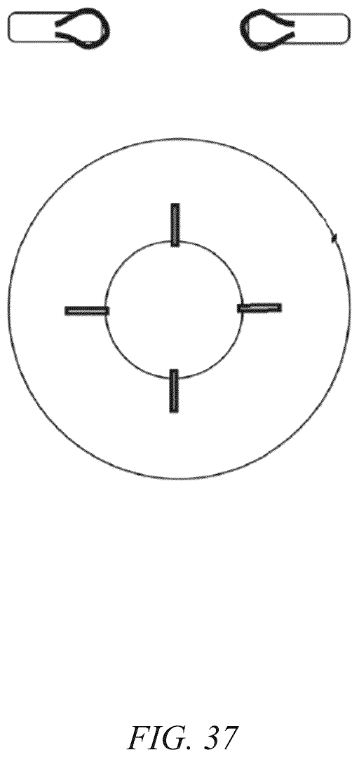

FIG. 37 depicts an embodiment of a clip secured grommet. The clip secured grommet may comprise one or more clips on the inner circumference of a hollow grommet. A one-sided grommet is sutured around the otomy, and the tissue surrounding the otomy is secured to the grommet with clips. The clips secure the tissue surrounding the otomy, reducing trauma and locomotion of the tissue to preserve the otomy.

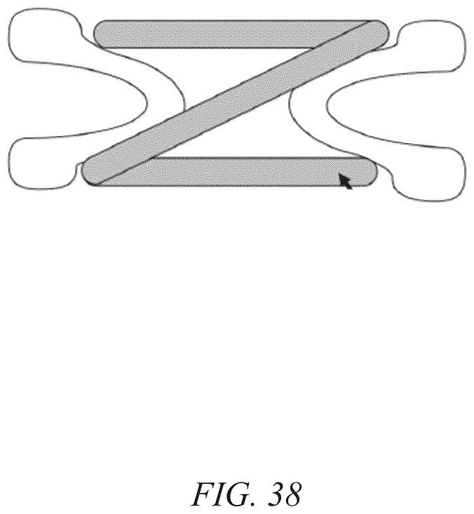

FIG. 38 depicts an embodiment of a coil compressed grommet. A grommet is stored in a delivery device in a compressed delivery configuration. The medical professional advances the delivery device through the otomy to the distal side of the tissue. The medical professional then pulls back on the delivery device, deploying the distal end of the grommet into the distal lumen. The distal end of the grommet expands into a deployed configuration. The medical professional then pulls back on the delivery device, deploying the proximal end of the grommet into the proximal lumen. The proximal end of the grommet expands into a deployed configuration. The grommet is then secured with a coil to preserve the otomy and secure the tissue surrounding the otomy.

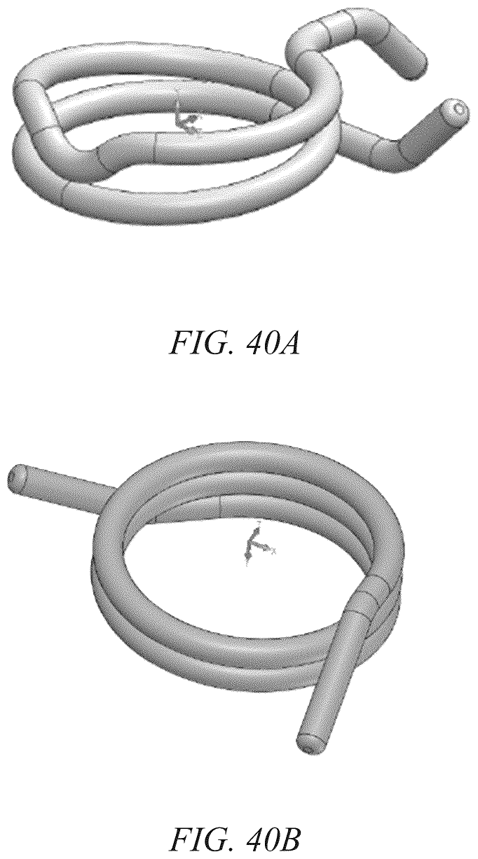

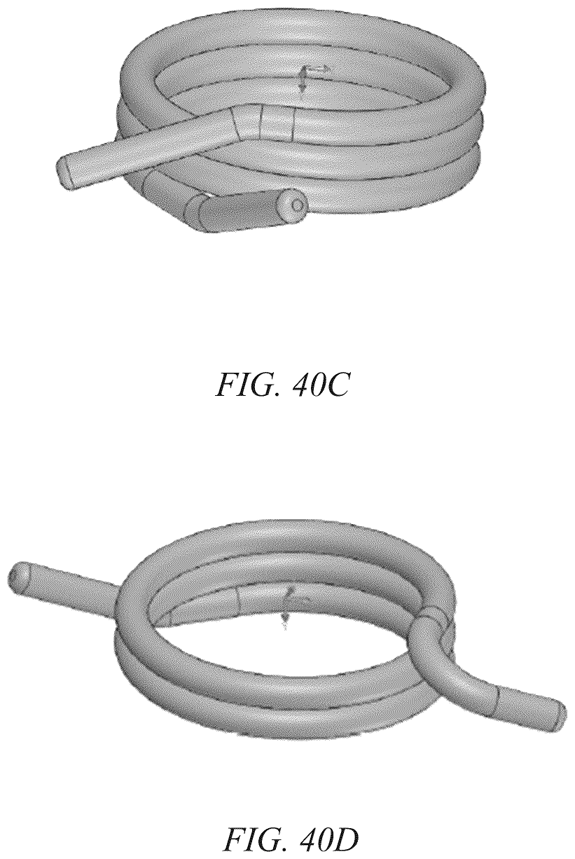

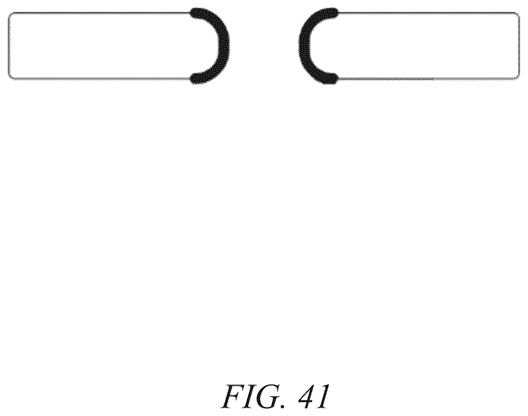

FIGS. 40A-40D depict illustrative embodiments of coiled wire otomy securement devices. A single length of nitinol, stainless steel, or other biocompatible material may be coiled and deployed around the otomy site to secure tissue. Layers of the coiled clamp tissue therebetween to secure the tissue and reduce trauma or locomotion of the tissue to secure the otomy. The hollow center of the coils allows for immediate passage of fluids and other materials through the otomy before creation of a permanent anastomosis.

FIG. 41 depicts an embodiment of an adhesive reinforced otomy. After creation of an otomy, the tissue surrounding the otomy is coated with an adhesive to reduce tears and/or stretching of the otomy site. Adhesive on the tissue leaves the center of the otomy site open, allowing for passage of fluid and other materials through the otomy, while preserving the integrity of the otomy site.

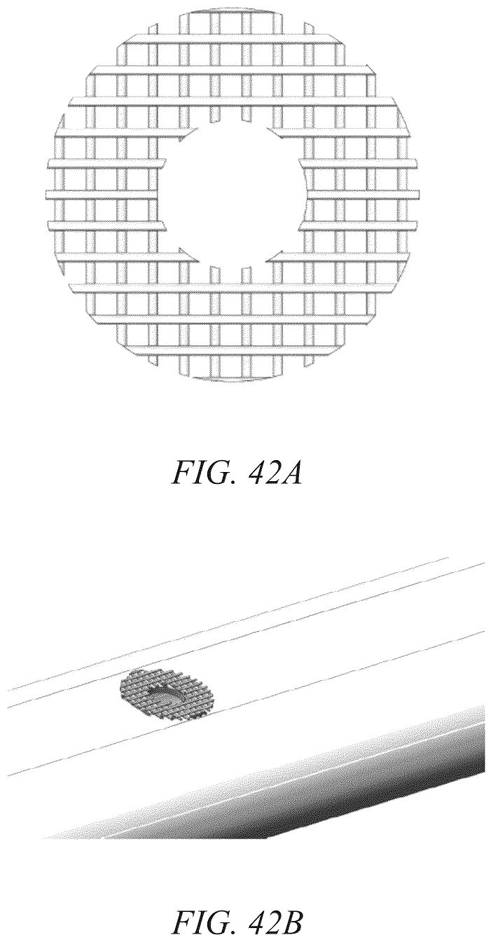

FIGS. 42A-42B depict an embodiment of a patch for securing an otomy. After creation of an otomy, the otomy site is supported by a patch adhered to the tissue surrounding the otomy site. The patch has an annular shape, allowing for passage of fluid and other materials through the patch and the otomy.

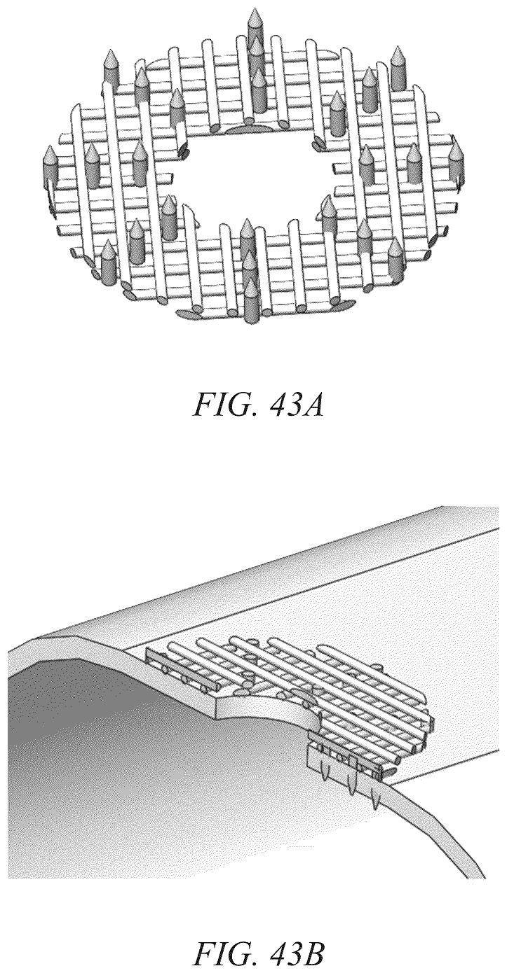

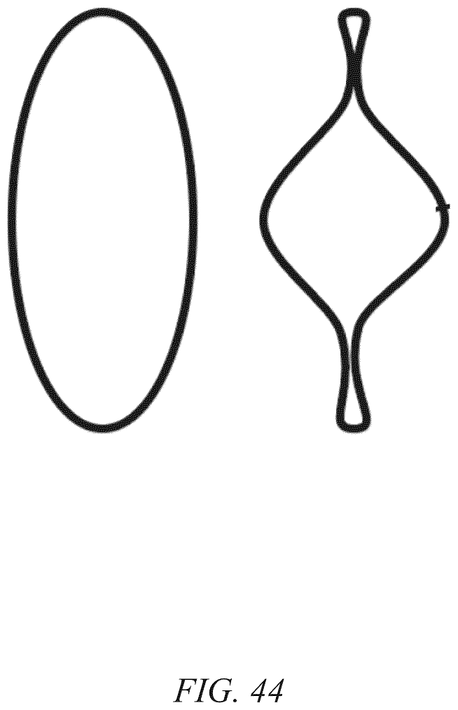

FIGS. 43A-43B depict an embodiment of a puncture patch for securing an otomy. After creation of an otomy, the otomy site is supported by a patch mechanically adhered to the tissue surrounding the otomy site. The patch may comprise spikes or other protrusions capable of puncturing the tissue surrounding the otomy site to secure the patch to the tissue, preserving the otomy.

FIG. 44 depicts an embodiment of a crimped wire form otomy clip. The crimped wire otomy clip is stored in an annular delivery configuration in a delivery device. The crimped wire otomy clip may be deployed prior to or subsequent to the creation of the otomy. On delivery, the crimped wire otomy is clipped into a deployed configuration. The crimps clamp tissue surrounding the otomy site, securing the tissue and supporting the otomy. The ring shape of the clip allows for immediate passage of fluid or other materials through the clip and the otomy.

The securing elements described above may be positioned on the target anatomy before creation of the otomy to serve as a guide for tool placement to create and access the otomy or after the creation of an otomy by means of adhesion, clamping, and/or encapsulating the anatomy. The element may be delivered by a tool or the surgeon to the target anatomy, and may remain within the body after completion of the procedure, or removed from the body prior to completion of the surgery. The devices described herein allow for control of an otomy site, reducing tissue tearing, dilation of the otomy, and unintended trauma to the site.

As mentioned above, in some embodiments, magnetic compression anastomosis devices and/or other devices can be deployed through a channel of the otomy control device such as for deployment in a distal lumen. The following is an exemplary anastomosis procedure using otomy control devices of the types described above to support otomies and deliver magnetic compression anastomosis devices, specifically in the context of single anastomosis duodenal ileostomy (SADI) with endo intracorporeal anastomosis (EIA).

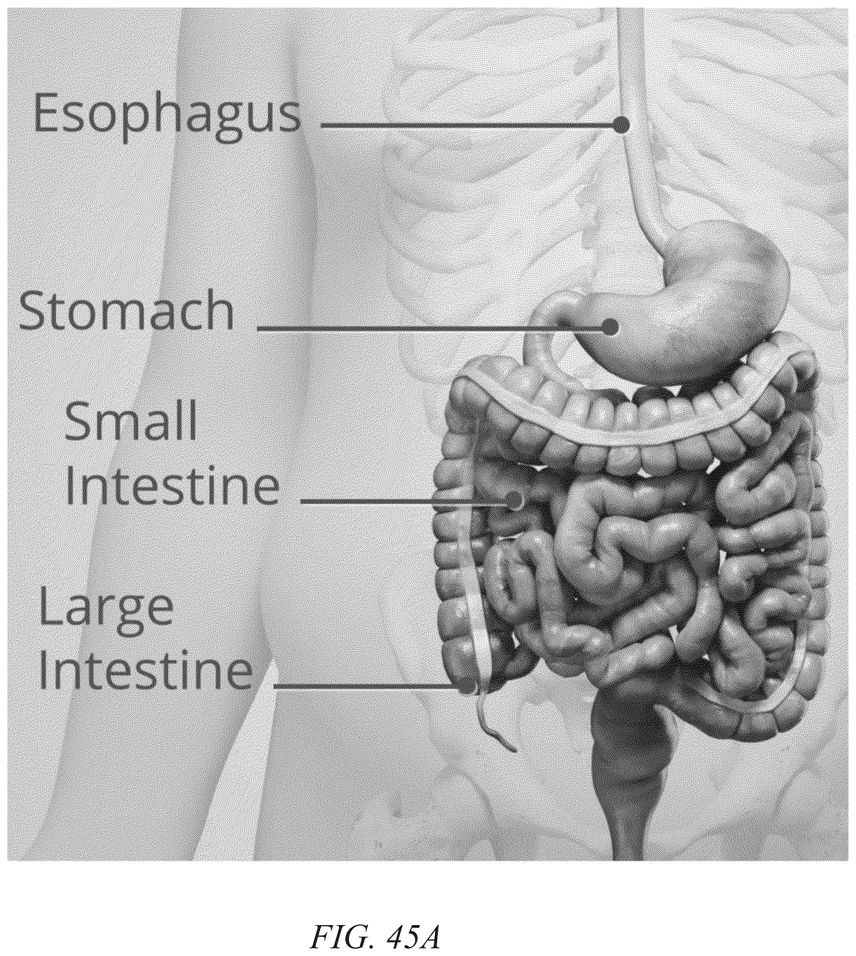

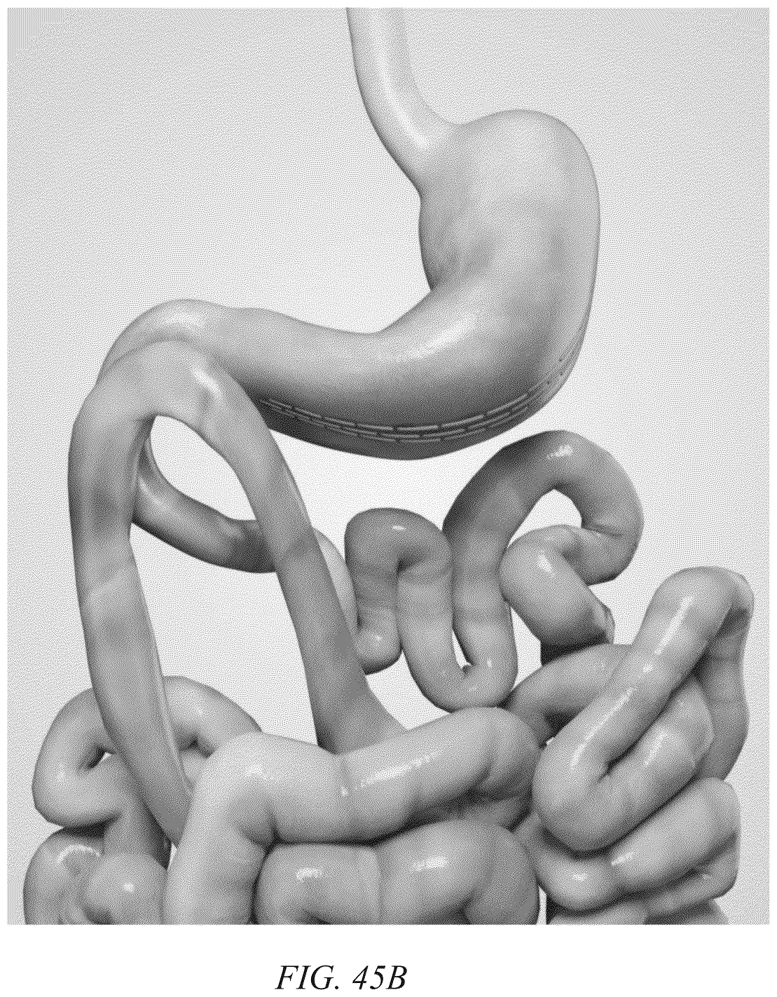

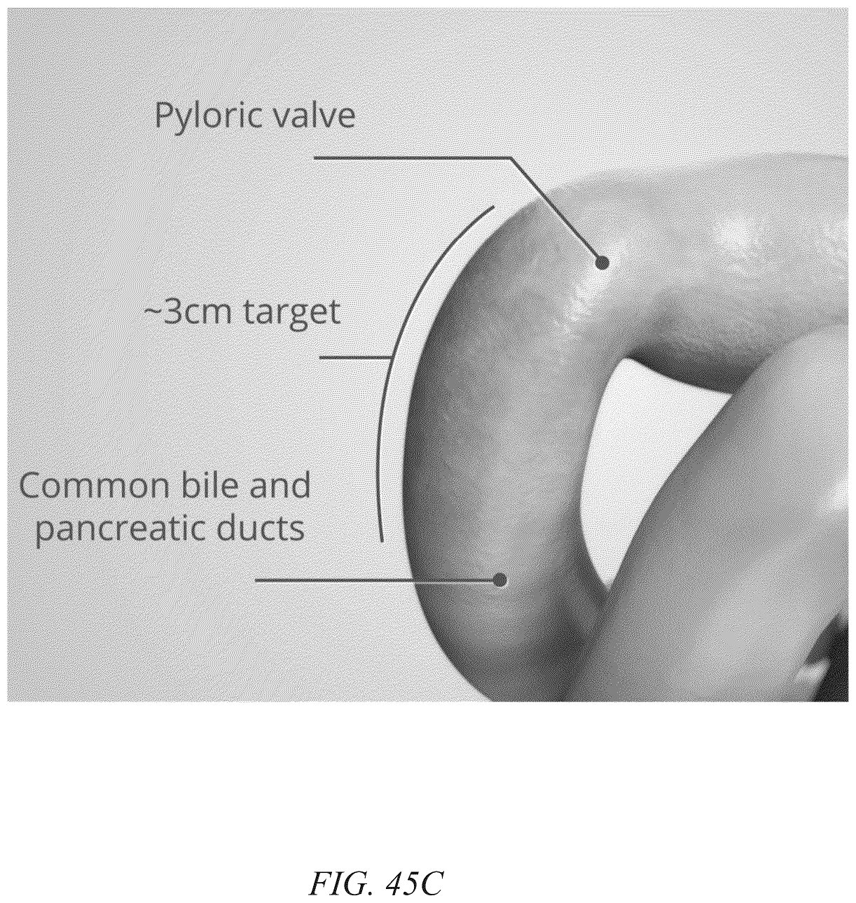

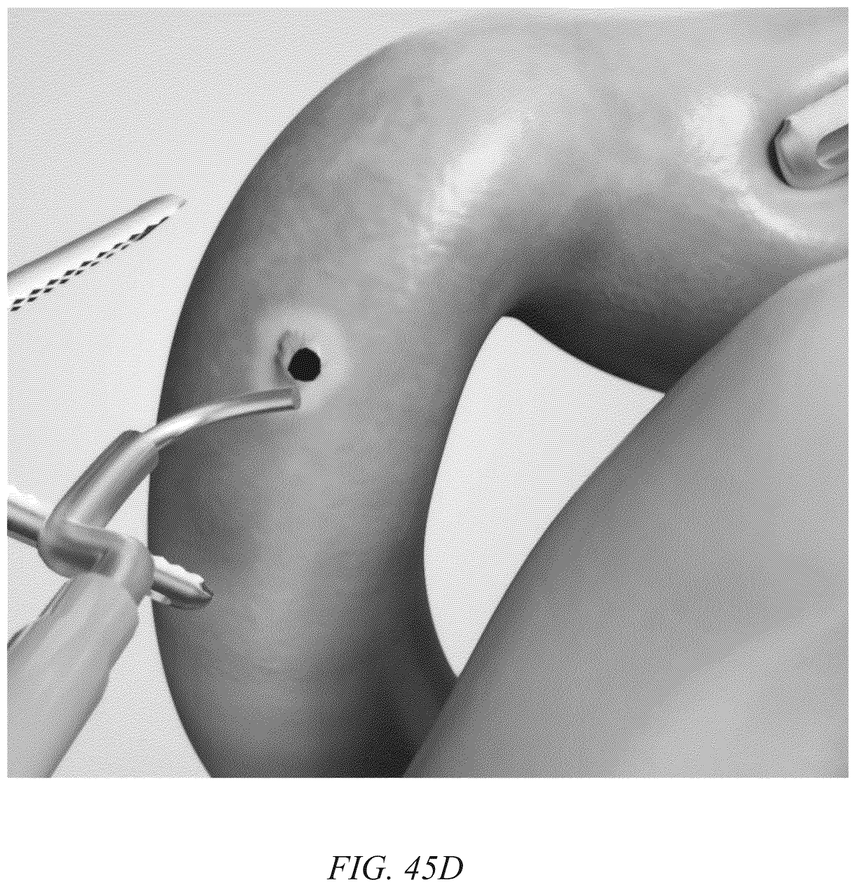

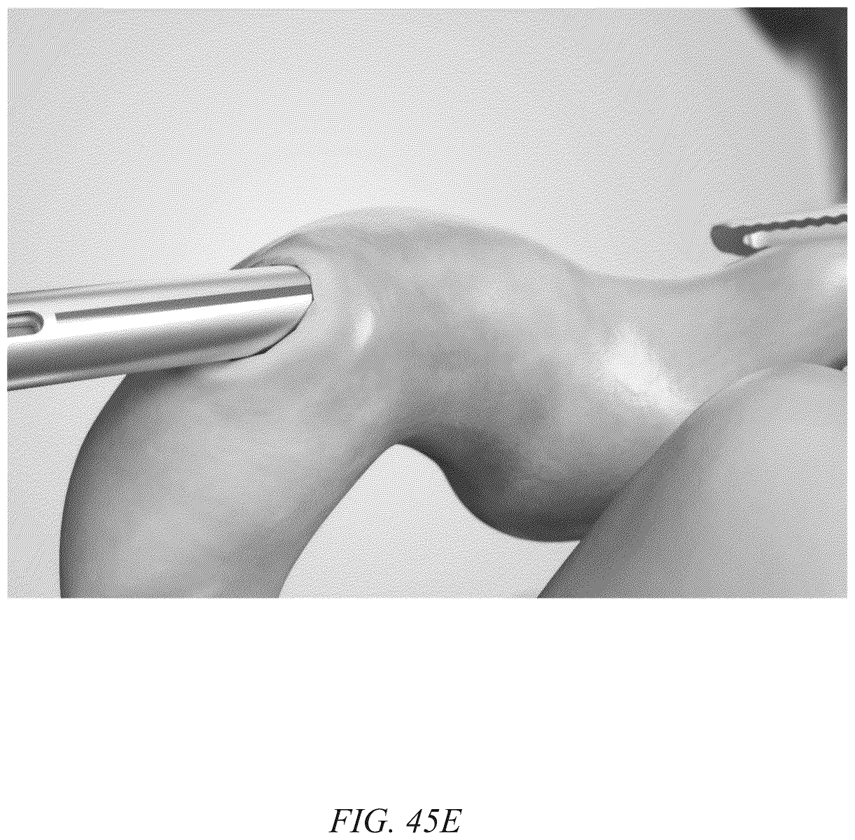

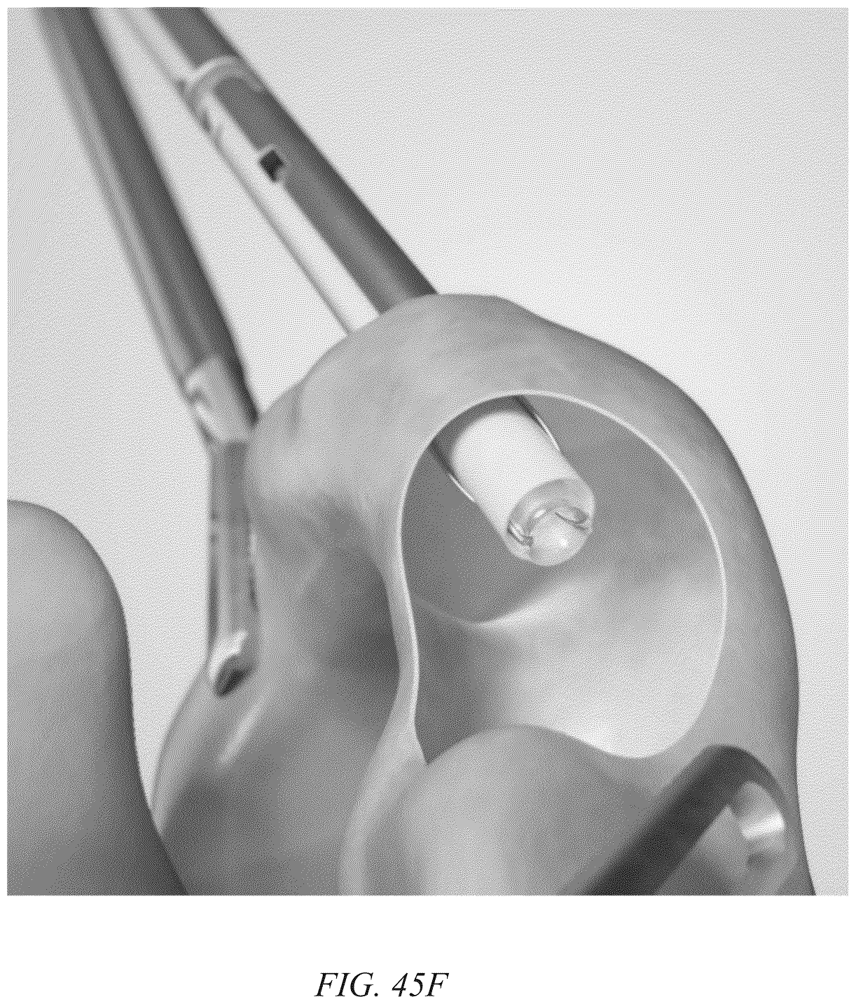

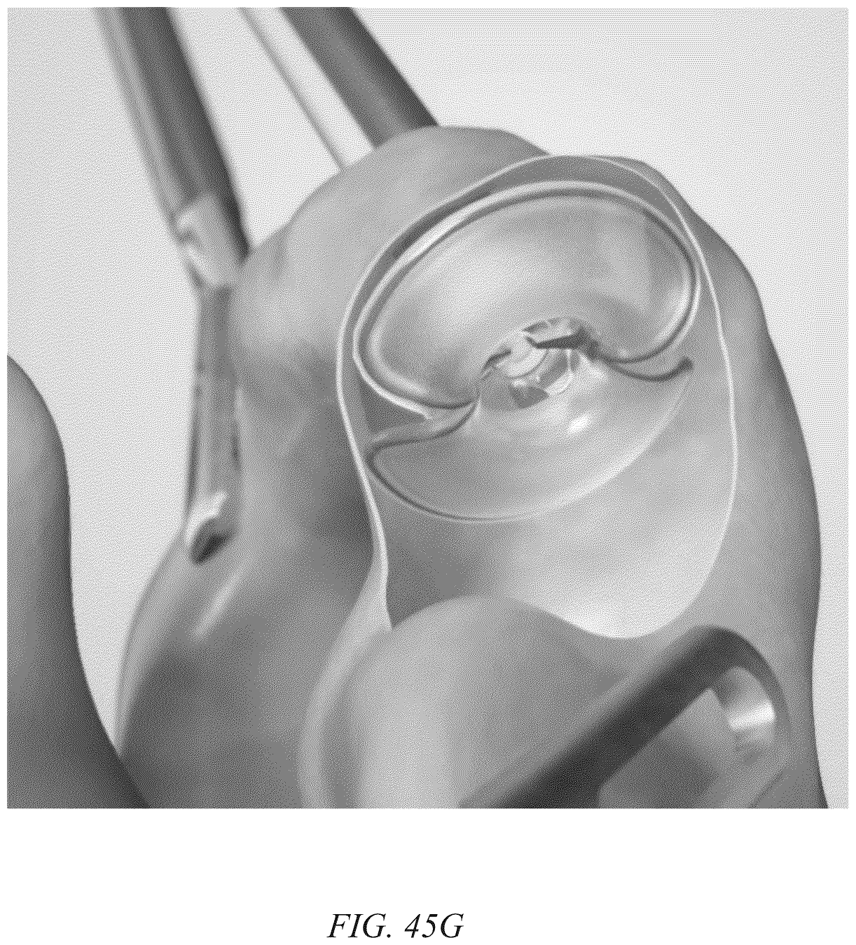

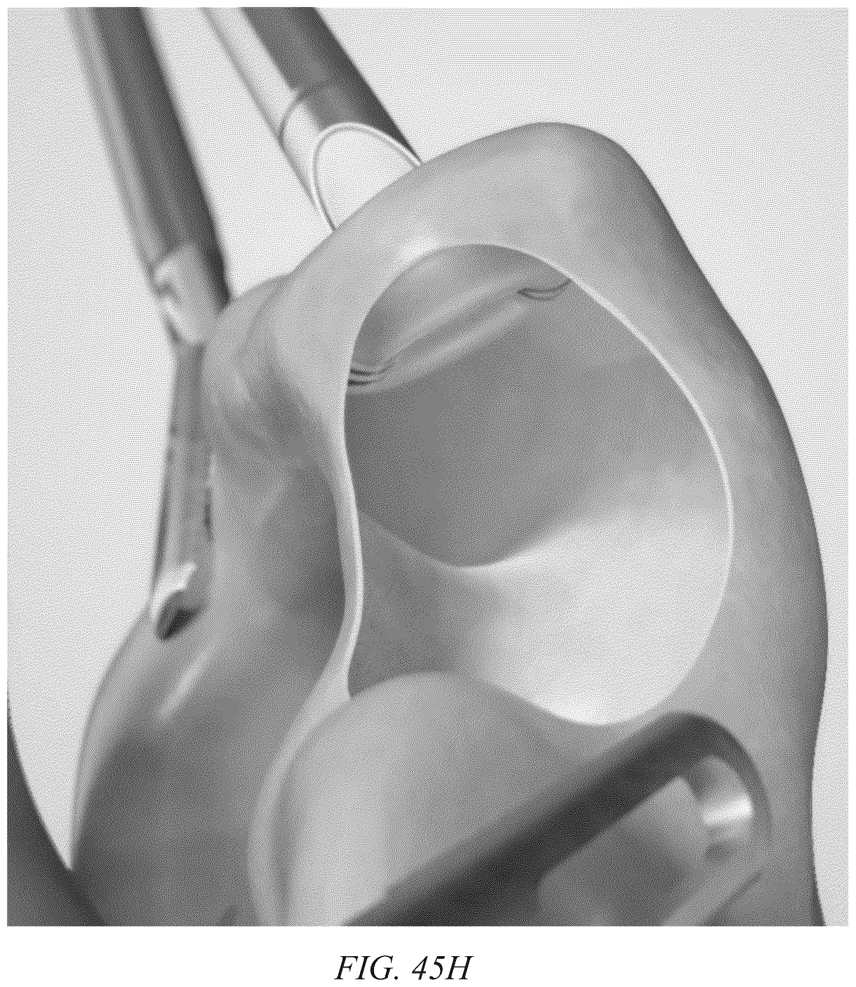

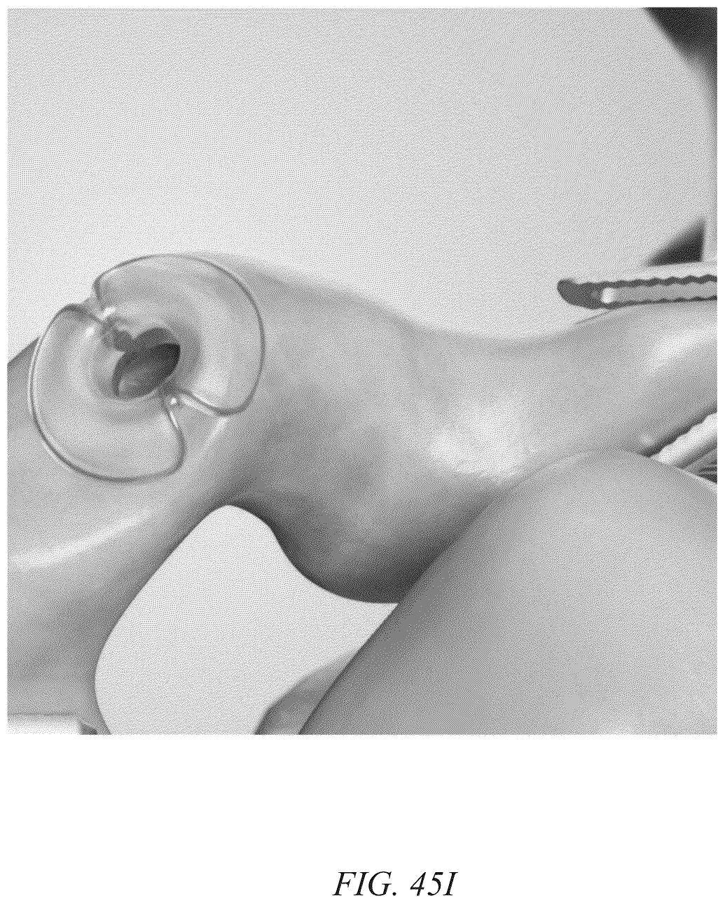

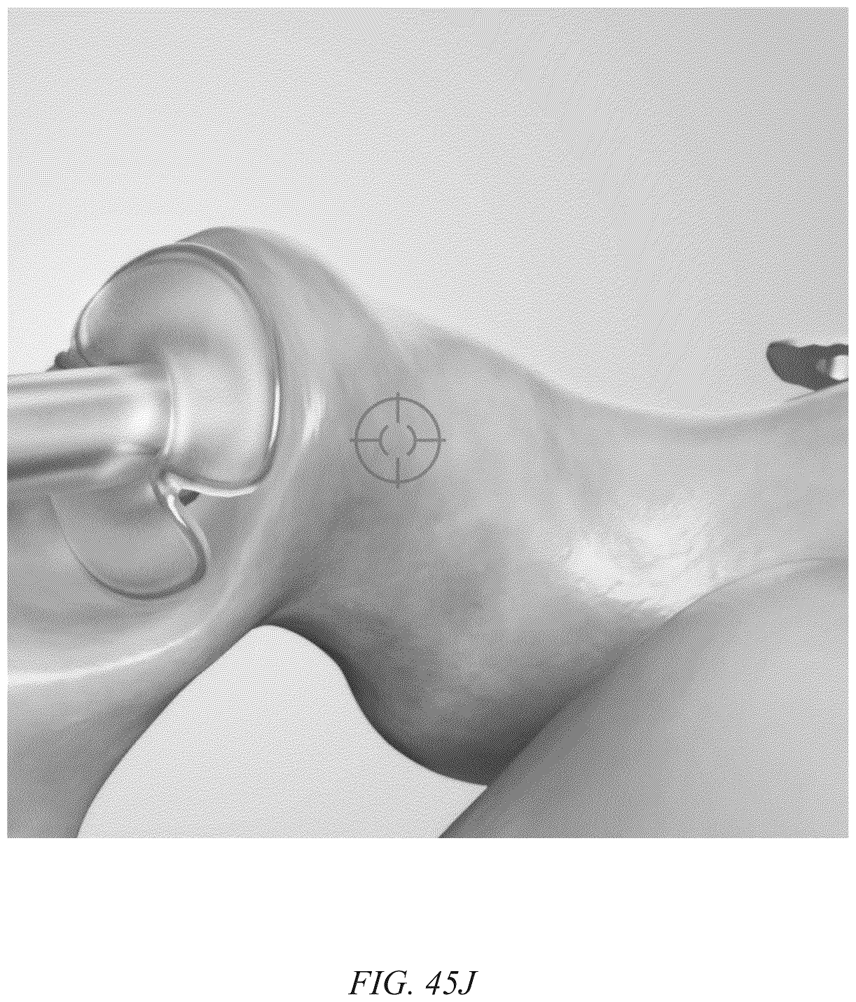

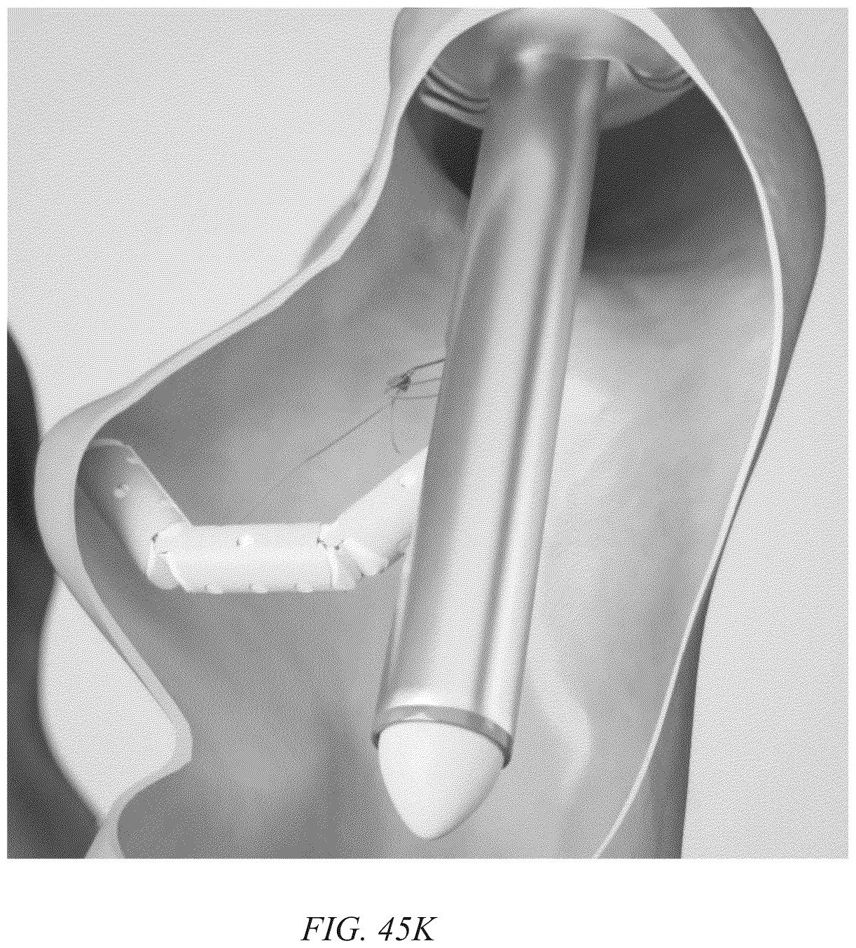

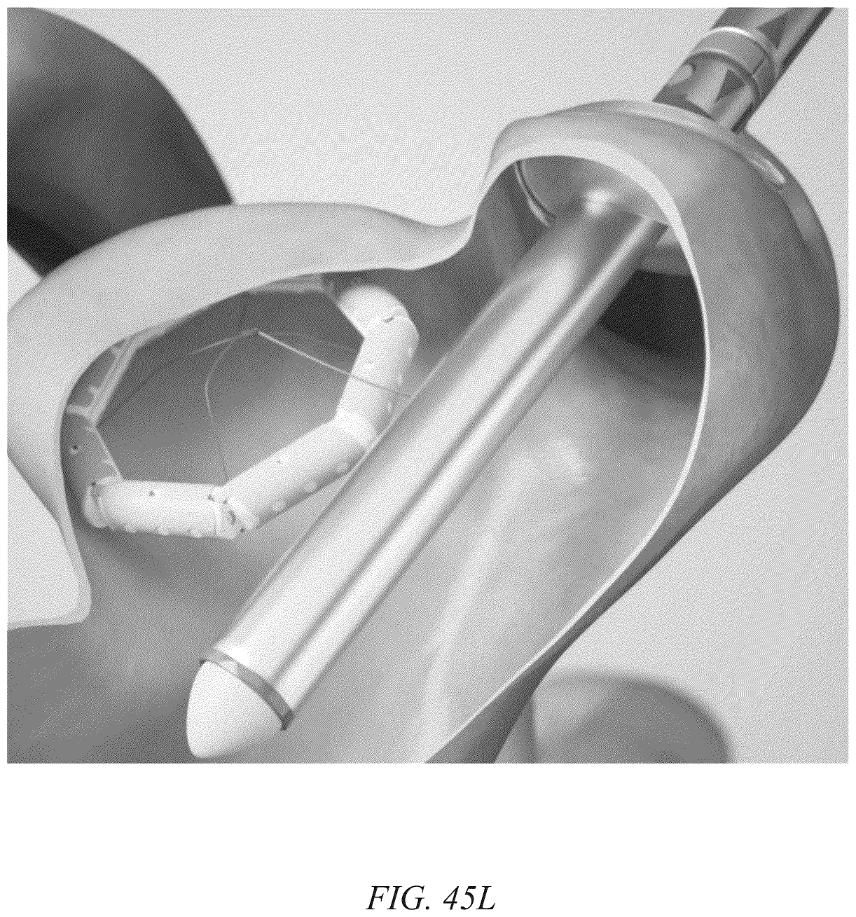

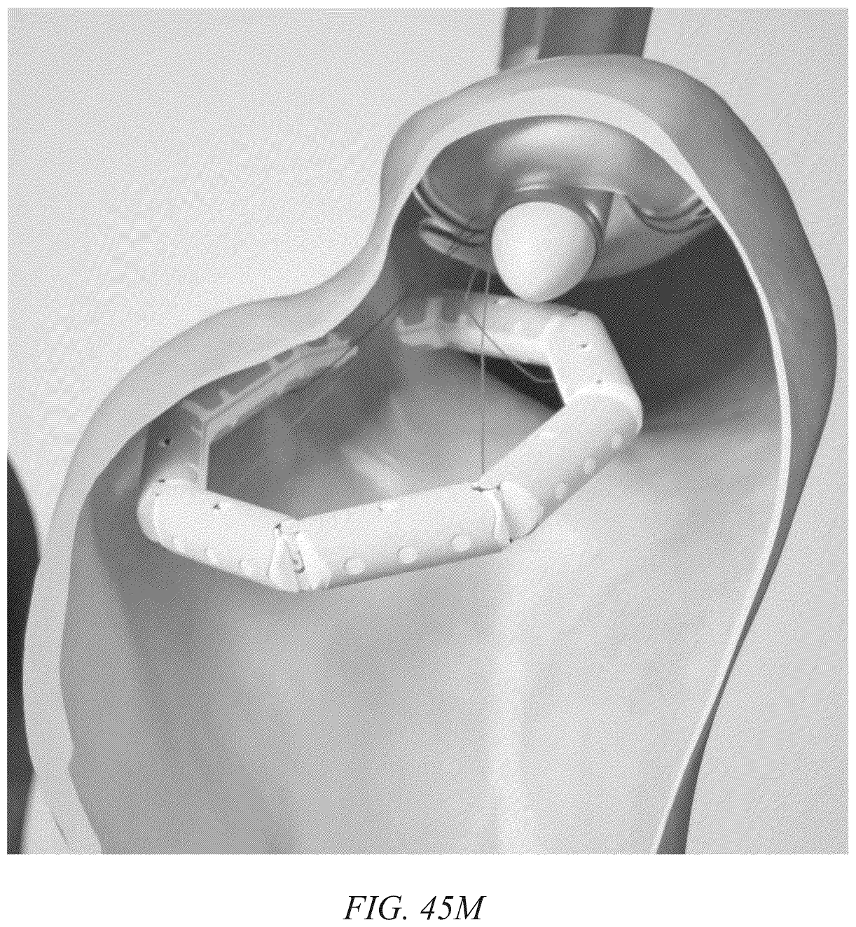

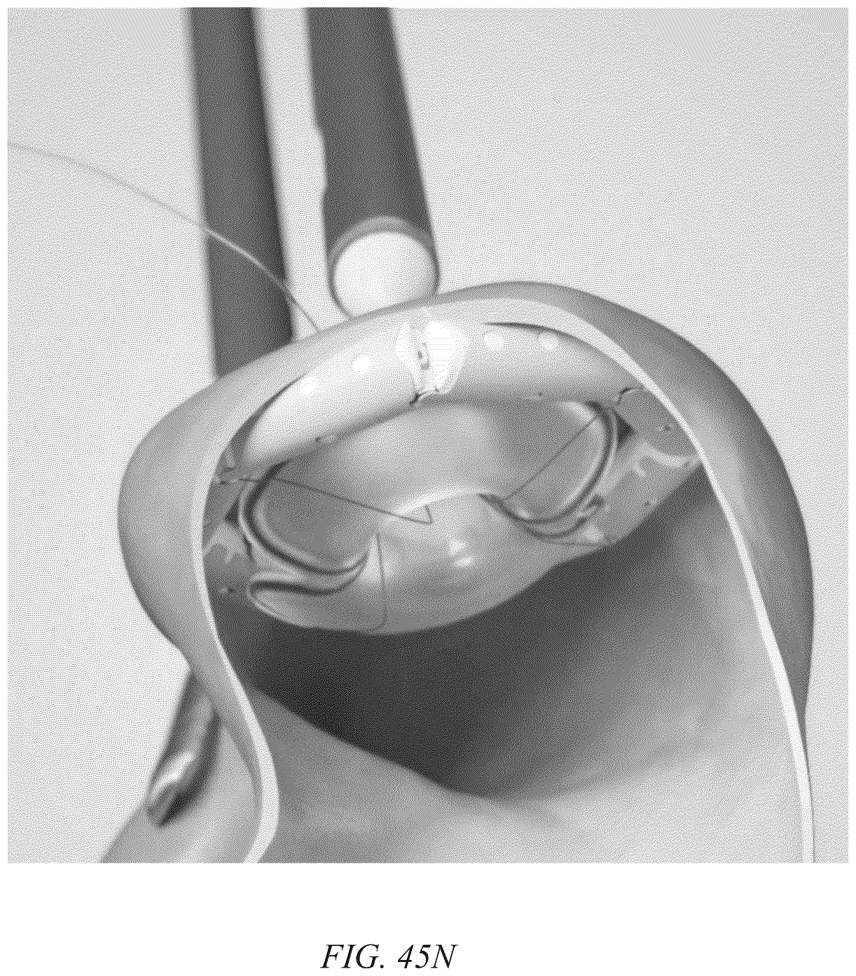

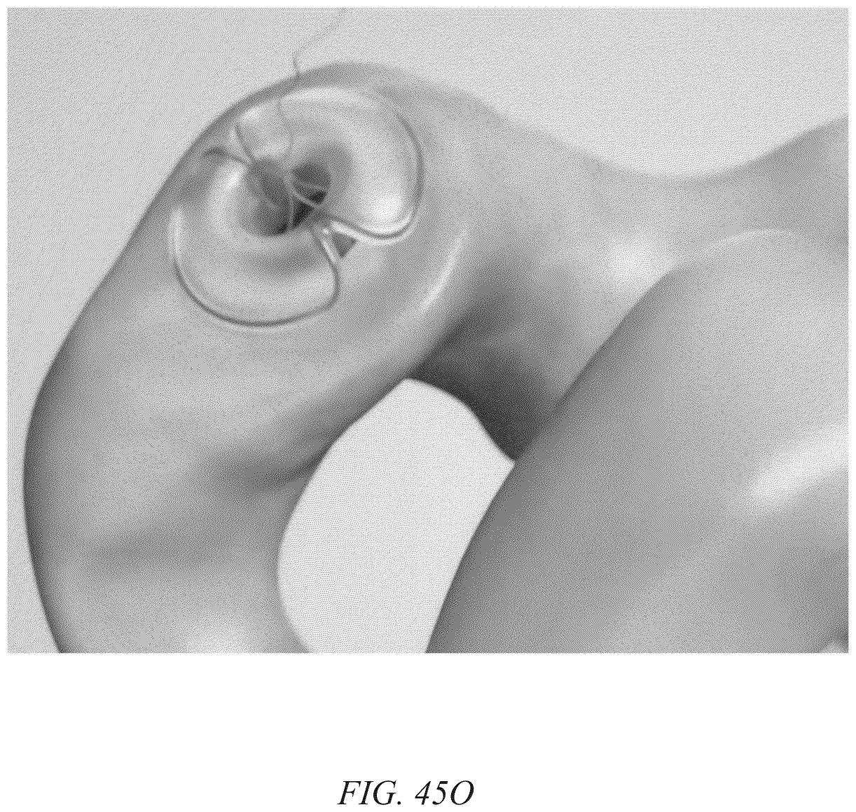

FIG. 45A is a schematic diagram showing relevant gastrointestinal anatomy for the exemplary procedure.

FIG. 45B is a schematic diagram showing the gastrointestinal anatomy upon completion of the procedure, i.e., with a sleeve gastrectomy of the stomach (e.g., reducing the size of the stomach to around 100-150 milliliters) and with a single anastomosis connecting the duodenum (e.g., approximately 3 centimeters beyond the stomach thereby preserving the pyloric valve) to the ileum (e.g., approximately 3 meters from the large intestine).

FIG. 45C is a schematic diagram showing the portion of the duodenum at which the anastomosis will be created.

FIG. 45D is a schematic diagram showing an otomy formed in the duodenum, e.g., using an electrocauterization device.

FIG. 45E is a schematic diagram showing insertion of a delivery device through the otomy and into the duodenum for delivery of an otomy control device.

FIG. 45F is a schematic diagram showing the delivery device with otomy control device from within the duodenum.

FIG. 45G is a schematic diagram showing deployment of the distal flange of the otomy control device within the duodenum such as by retracting the delivery device and/or advancing a pusher device within the delivery device. Note here that this particular otomy control device includes an inner framework and an external covering.

FIG. 45H is a schematic diagram showing retraction of the delivery device in preparation for delivery of the proximal flange of the otomy control device.

FIG. 45I is a schematic diagram showing delivery of the proximal flange of the otomy control device on the outside of the duodenum such that the otomy is not controlled from both inside and outside of the duodenum.

With the otomy so controlled, one or more devices such as a magnetic compression anastomosis device can be placed within the duodenum through the channel in the otomy control device.

FIG. 45J is a schematic diagram showing an instrument inserted into the duodenum through the channel of the otomy control device. In this example, the instrument is a delivery device for delivering a self-assembling magnetic compression anastomosis device.

FIG. 45K is a schematic diagram showing the instrument within the duodenum delivering the self-assembling magnetic compression anastomosis device. In this image, the magnetic compression anastomosis device is partially deployed. Sutures used to manipulate the magnetic compression anastomosis device can be seen.

FIG. 45L is a schematic diagram showing the self-assembling magnetic compression anastomosis device fully deployed and fully assembled within the duodenum. Sutures used to manipulate the magnetic compression anastomosis device can be seen.

FIG. 45M is a schematic diagram showing the instrument being retracted from the duodenum to leave the fully deployed magnetic compression anastomosis device and sutures.

FIG. 45N is a schematic diagram showing the magnetic compression anastomosis device manipulated using the sutures into position around the otomy control device against the inside surface of the duodenum surrounding the otomy and otomy control device. In this embodiment, the flange of the otomy control device is shaped or otherwise configured to assist with positioning of the magnetic compression anastomosis device around the otomy control device, e.g., a tapered configuration.

FIG. 45O is a schematic diagram showing the completed duodenal otomy from the external surface of the duodenum.

A similar procedure can be performed for the ileum either before or after the otomy is formed in the duodenum.

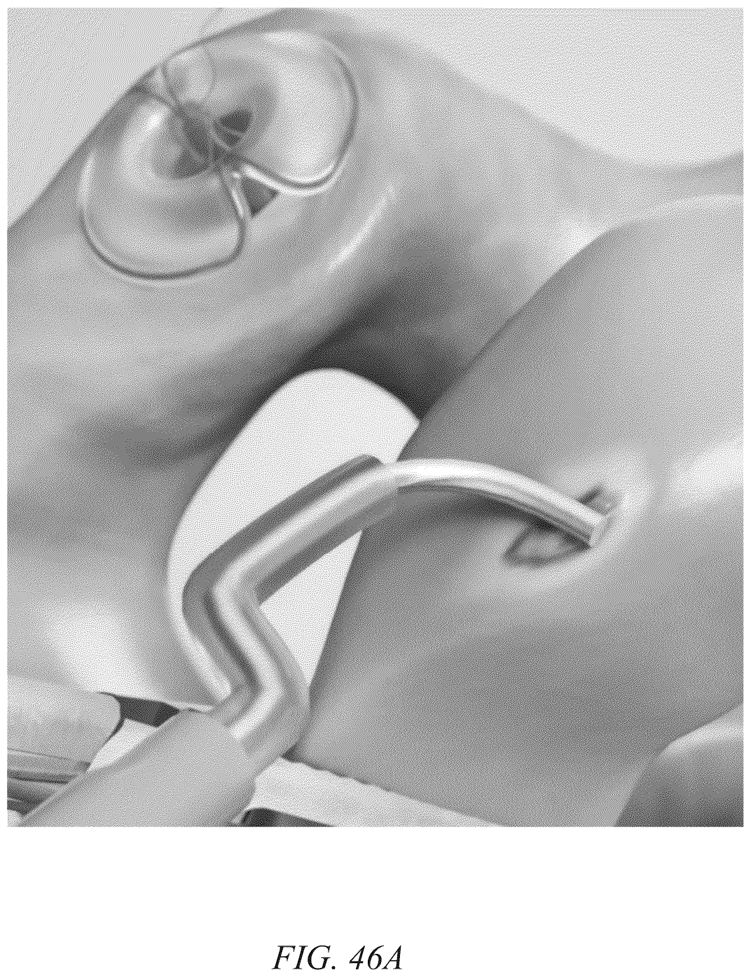

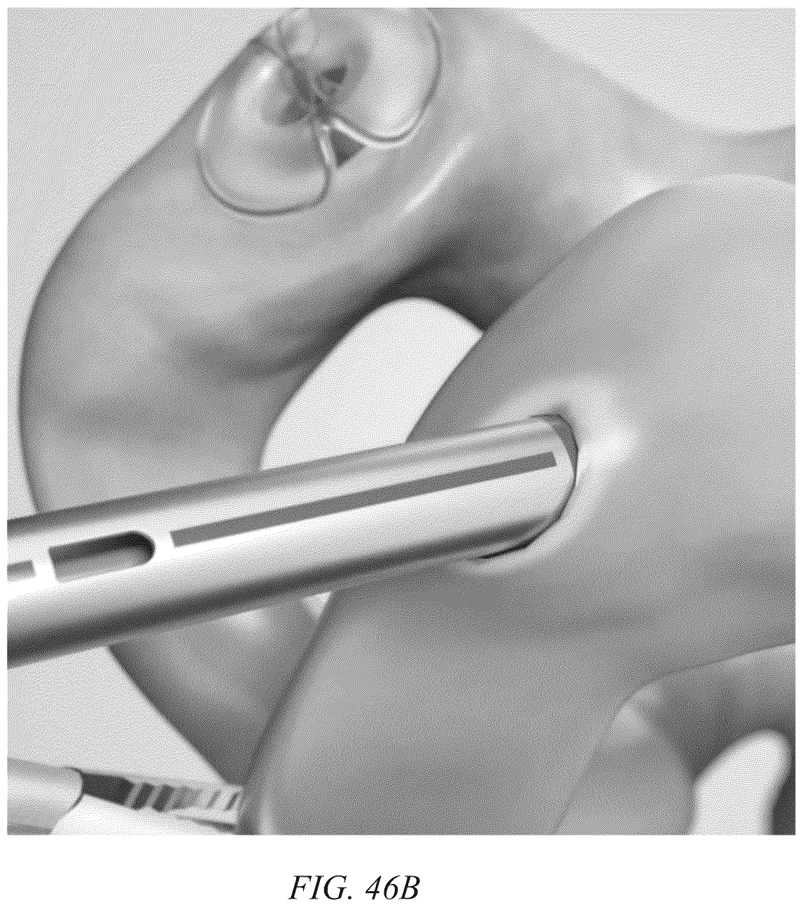

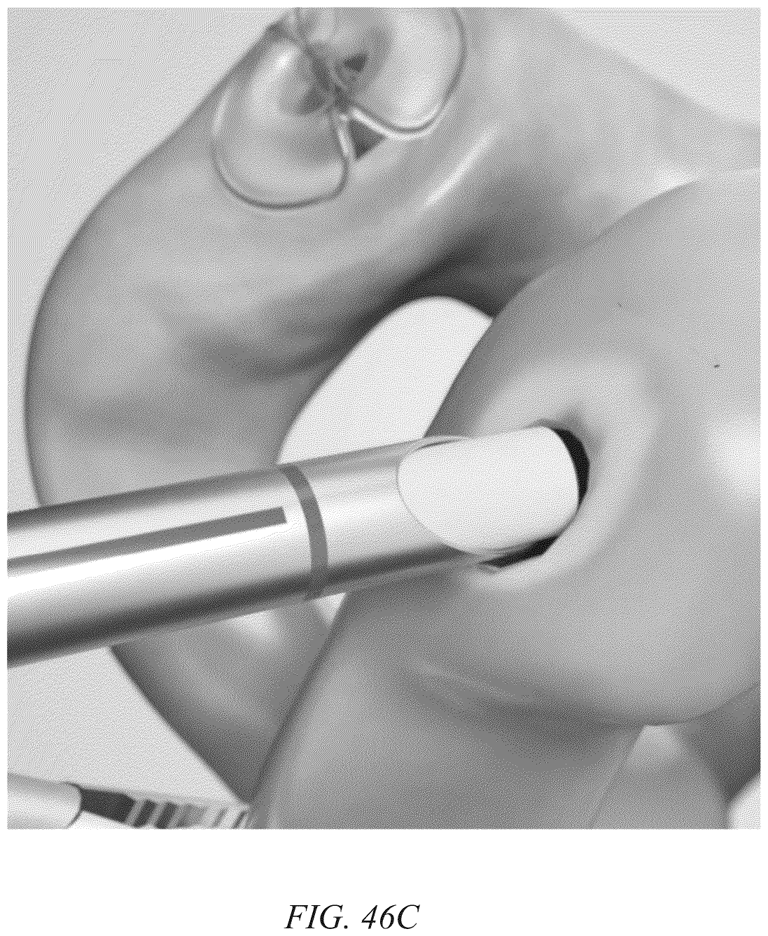

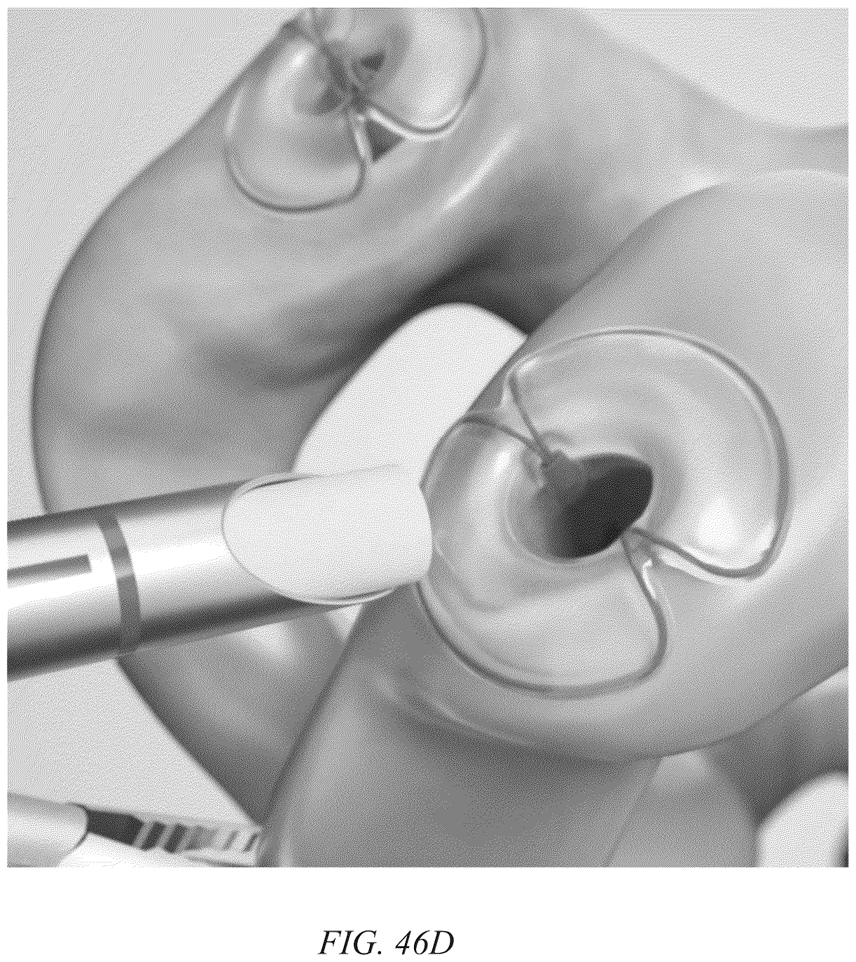

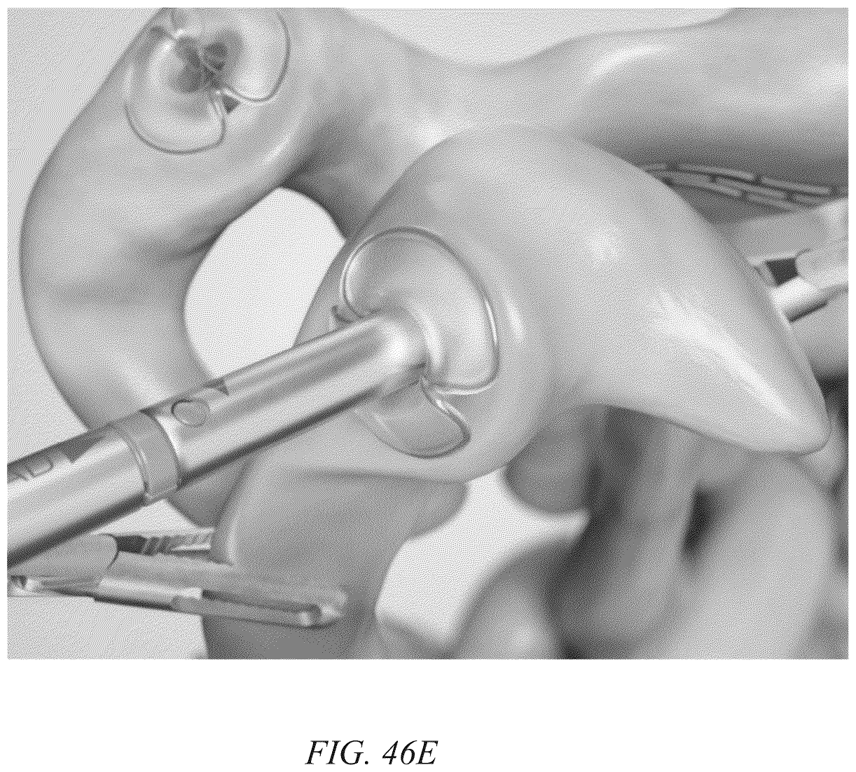

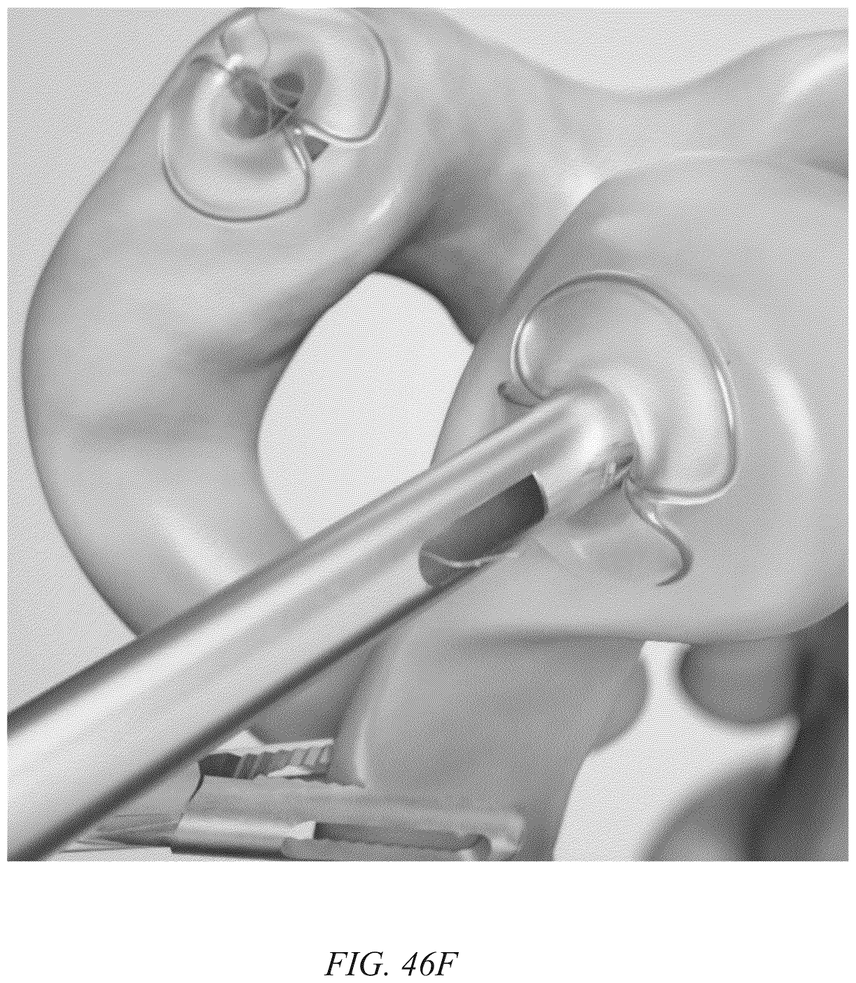

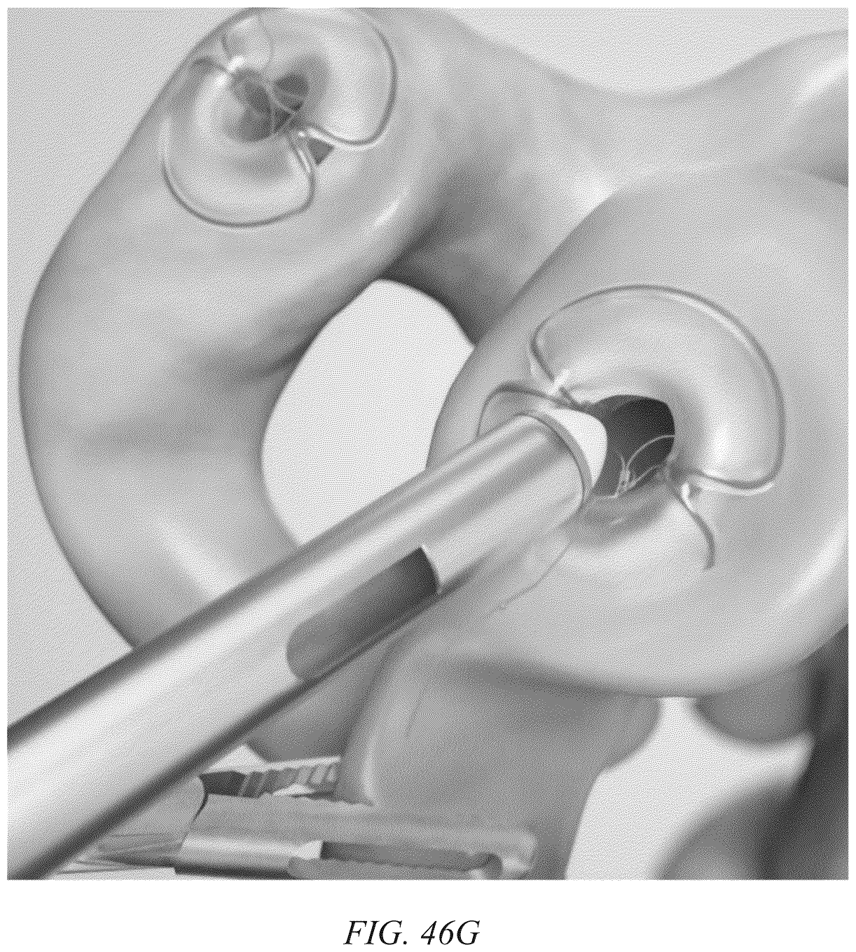

FIG. 46A is a schematic diagram showing an otomy formed in the ileum, e.g., using an electrocauterization device.

FIG. 46B is a schematic diagram showing insertion of a delivery device through the otomy and into the ileum for delivery of an otomy control device.

FIG. 46C is a schematic diagram showing retraction of the delivery device from the ileum after deployment of the distal flange of the otomy control device within the ileum.

FIG. 46D is a schematic diagram showing the proximal flange of the otomy control device delivered to the external surface of the ileum.

FIG. 46E is a schematic diagram showing an instrument inserted into the ileum through the channel of the otomy control device. In this example, the instrument is a delivery device for delivering a self-assembling magnetic compression anastomosis device.

FIG. 46F is a schematic diagram showing the instrument following delivery of the magnetic compression anastomosis device within the ileum. The sutures used to manipulate the magnetic compression anastomosis device can be seen.

FIG. 46G is a schematic diagram showing retraction of the instrument from the otomy to leave the self-assembling magnetic compression anastomosis device fully deployed and fully assembled within the ileum. Sutures used to manipulate the magnetic compression anastomosis device can be seen.

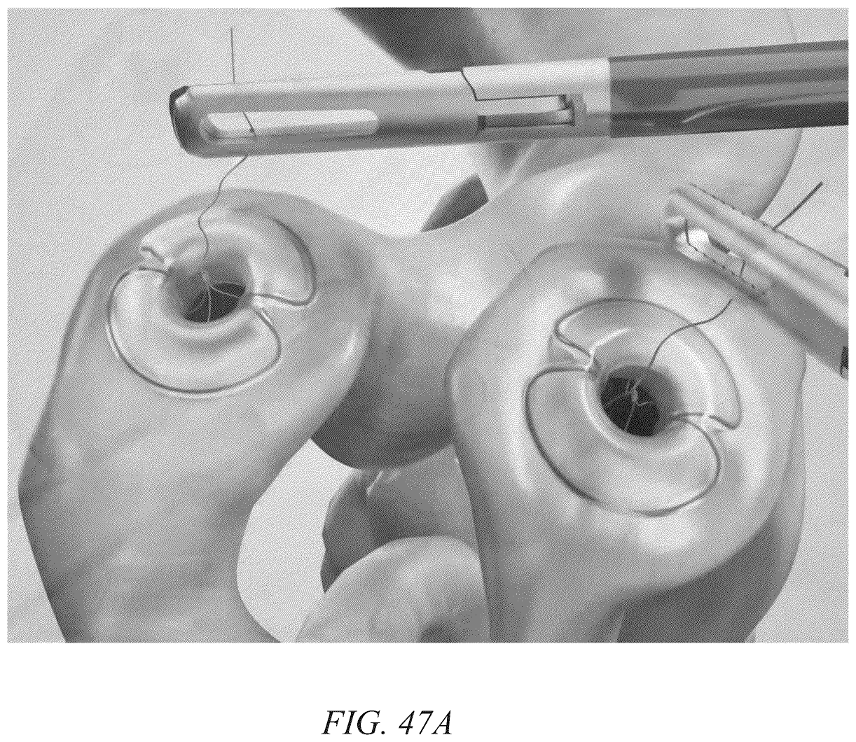

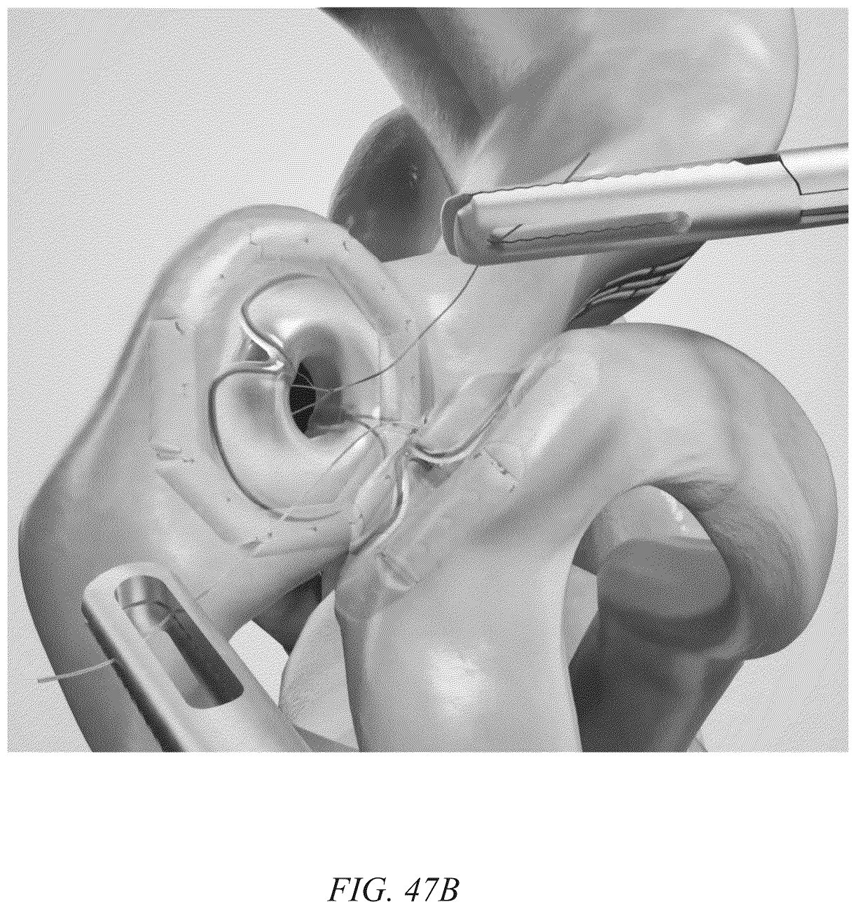

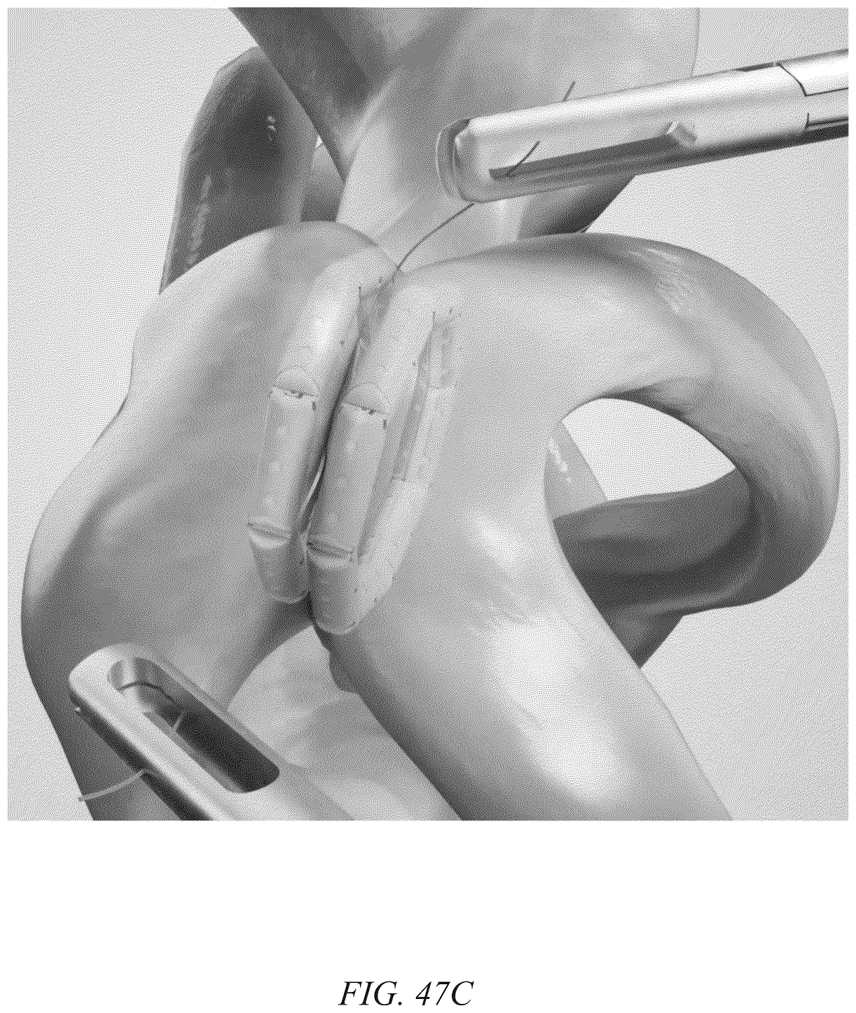

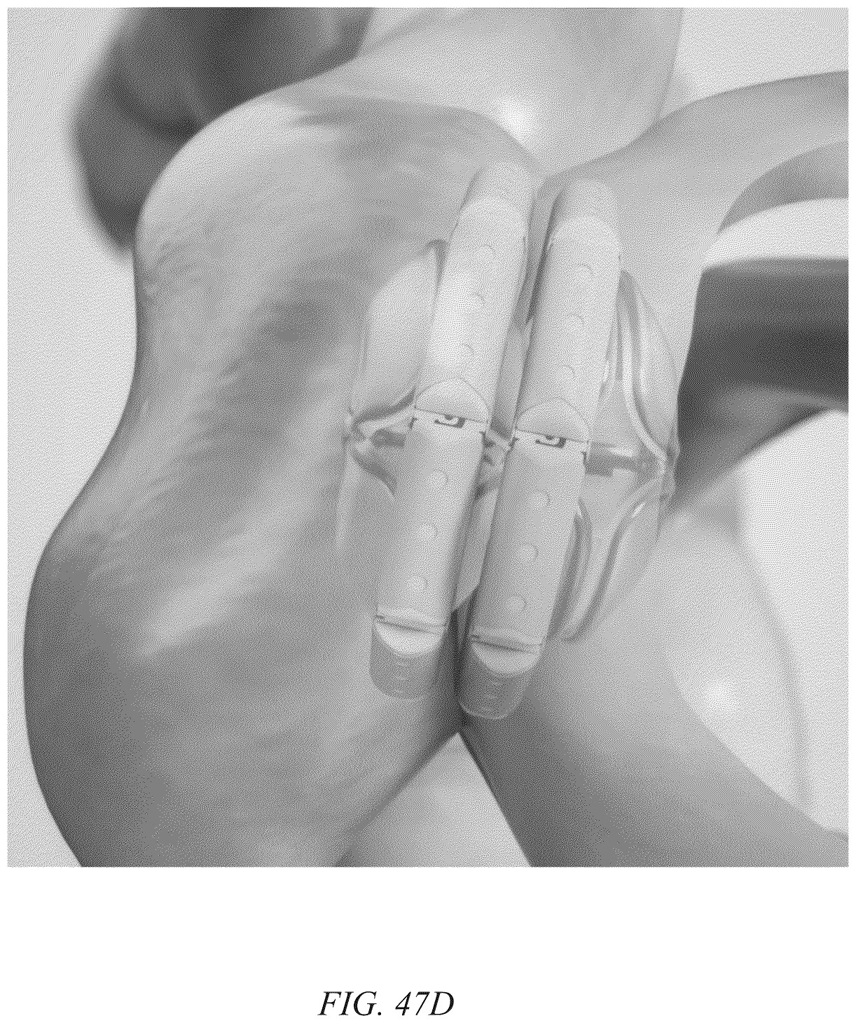

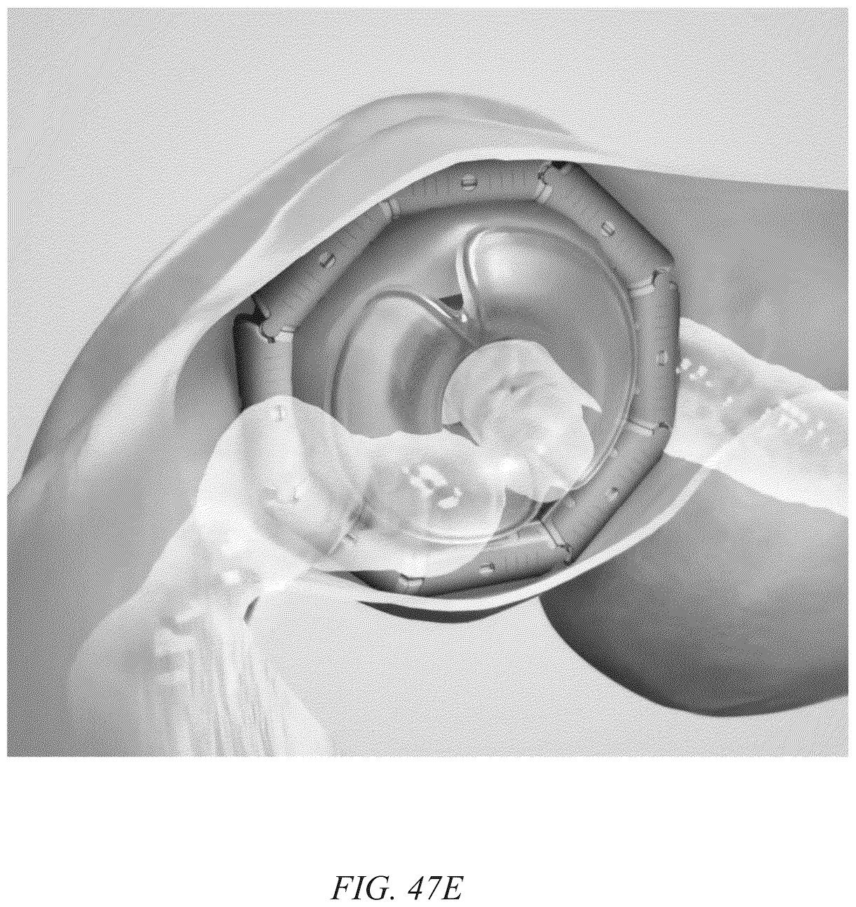

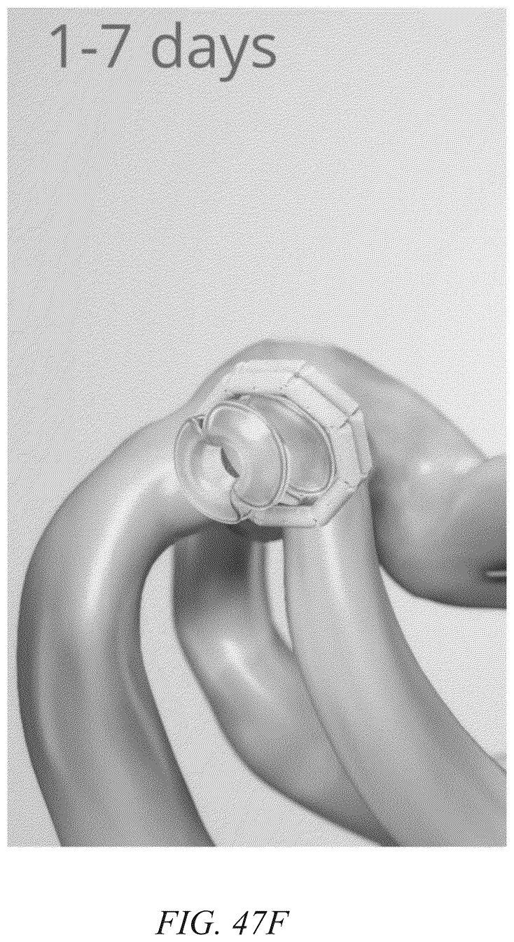

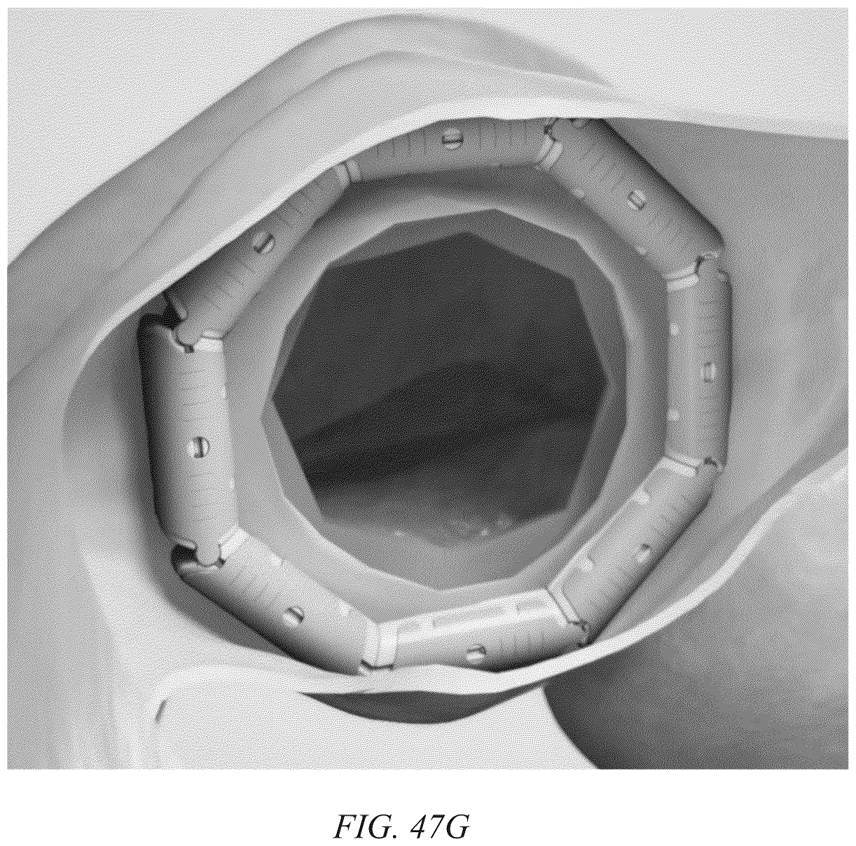

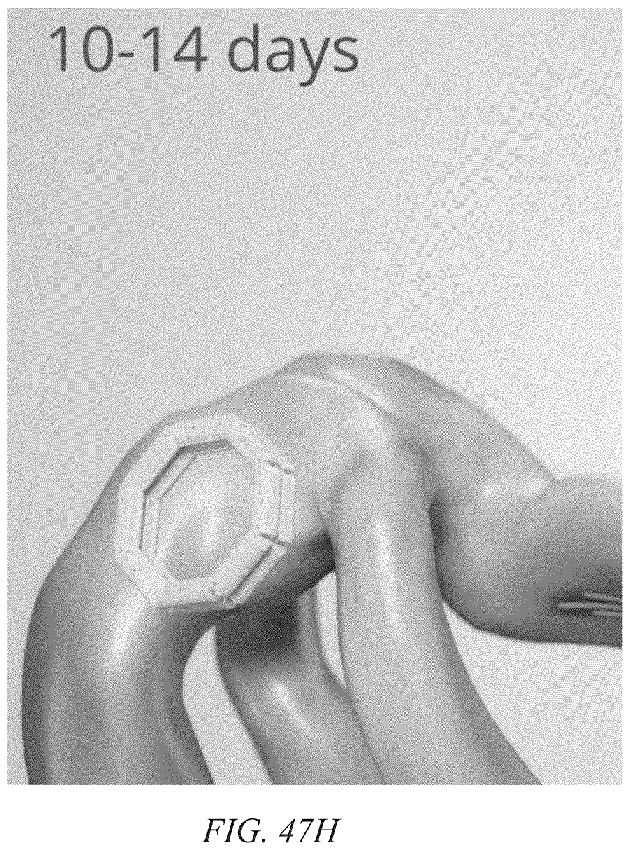

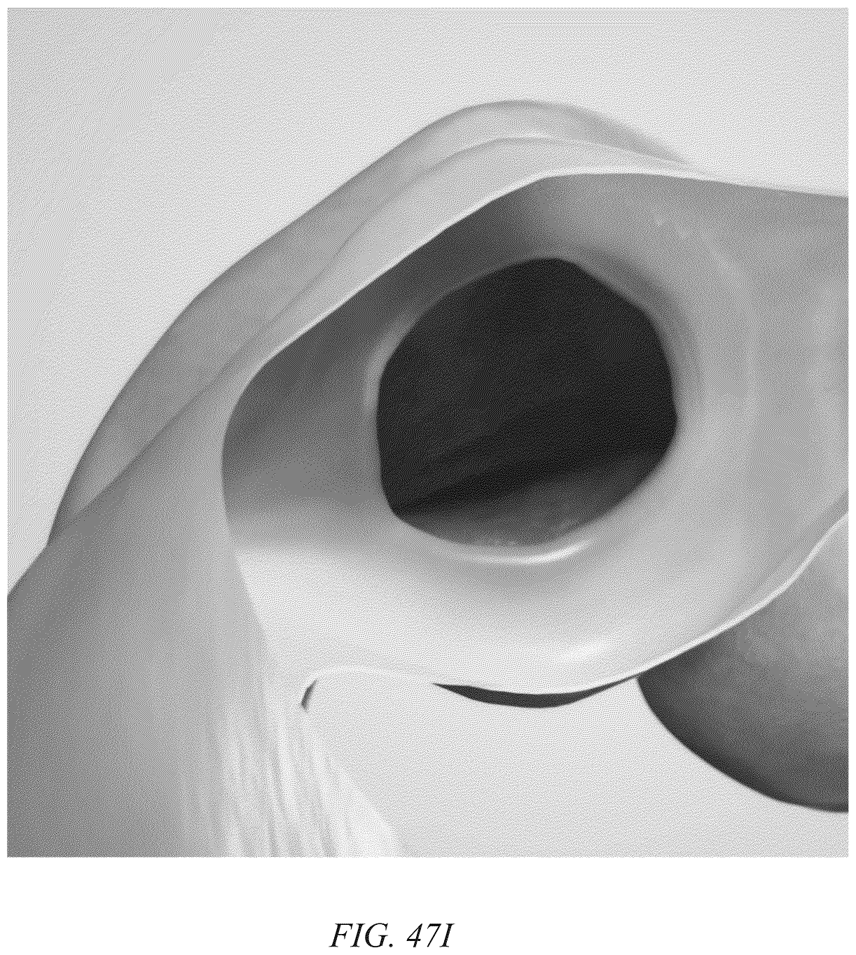

With the otomies fully formed and controlled in both the duodenum and ileum and with the magnetic compression anastomosis devices fully deployed within the duodenum and ileum, as depicted schematically in FIG. 47A, the two otomies can be brought together, as depicted schematically in FIG. 47B until they are in contact and aligned via the otomy control devices and magnetic compression anastomosis devices, as depicted schematically in FIG. 47C. The sutures can be used to position the magnetic compression anastomosis devices. FIG. 47D is a schematic diagram showing the otomy control devices and magnetic compression anastomosis devices positioned to allow for immediate fluidic communication from the duodenum to the ileum through the otomies, as depicted schematically in FIG. 47E, even before the anastomosis is formed. Typically after around 1-7 days, the tissues of the duodenum and ileum about the otomies will degrade to the point where the otomy control devices will dislodge and pass through the ileum to leave only the magnetic compression anastomosis device in place, as depicted schematically in FIG. 47F and FIG. 47G. Typically after around 10-14 days, the anastomosis will be fully formed and the magnetic compression anastomosis devices will dislodge and pass through the ileum to leave the fully formed anastomosis, as depicted schematically in FIG. 47H and FIG. 47I.

It should be noted that, in various alternative embodiments, the proximal and distal flanges may be the same configuration or may be different configurations, e.g., the distal flange that is positioned within the lumen may be shaped or otherwise configured to assist with positioning of a magnetic compression anastomosis device whereas the proximal flange that is positioned on the outside of the lumen may be shaped or otherwise configured to assist with joining of the two otomies as depicted schematically in FIGS. 47C-47D (e.g., the distal flange may be tapered while the proximal flange may be flatter in order to assist with mating of the two otomies). In this regard, the proximal may be shaped or configured to mate with another proximal flange, e.g., having raised and/or recessed features that interlock.

It should be noted that any two-piece otomy control device may include a ratcheting mechanism (e.g., as depicted schematically in FIGS. 27A-27B) or other connection mechanism to help maintain connection between the two halves.

It should be noted that the flanges of an otomy control device may have any number of loops or lobes, e.g., the device shown in FIG. 1 includes a single loop or lobe, the device shown in FIG. 45G includes two loops or lobes per flange, the device shown in FIG. 6A includes four loops or lobes per flange, the device shown in FIG. 5 includes 8 loops or lobes per flange, the device shown in FIG. 14A includes 16 loops or lobes per flange, etc., and devices can have any number of loops or lobes per flange, e.g., 1, 2, 3, 4, etc. It should be noted that the flanges can have the same number and configuration of loops or lobes or can have different numbers and/or configurations of loops or lobes. For example, the distal flange could have a plurality of loops or lobes (e.g., to facilitate delivery of the otomy control device and placement of the magnetic compression anastomosis device) and the proximal flange could have a single loop or lobe (e.g., to facilitate mating with the proximal flange of another otomy control device).

The following is a description of some alternative types of otomy control devices that operate generally by clamping around the otomy boundary.

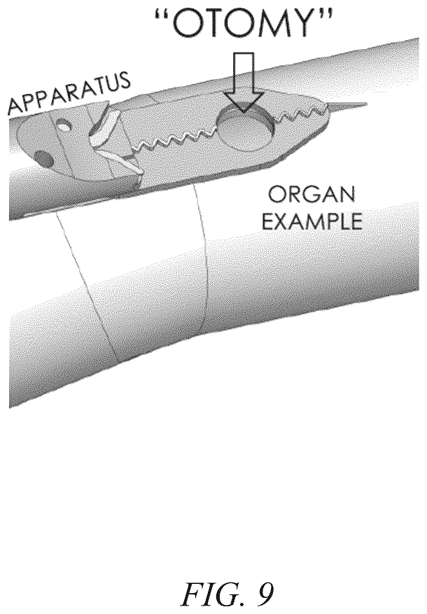

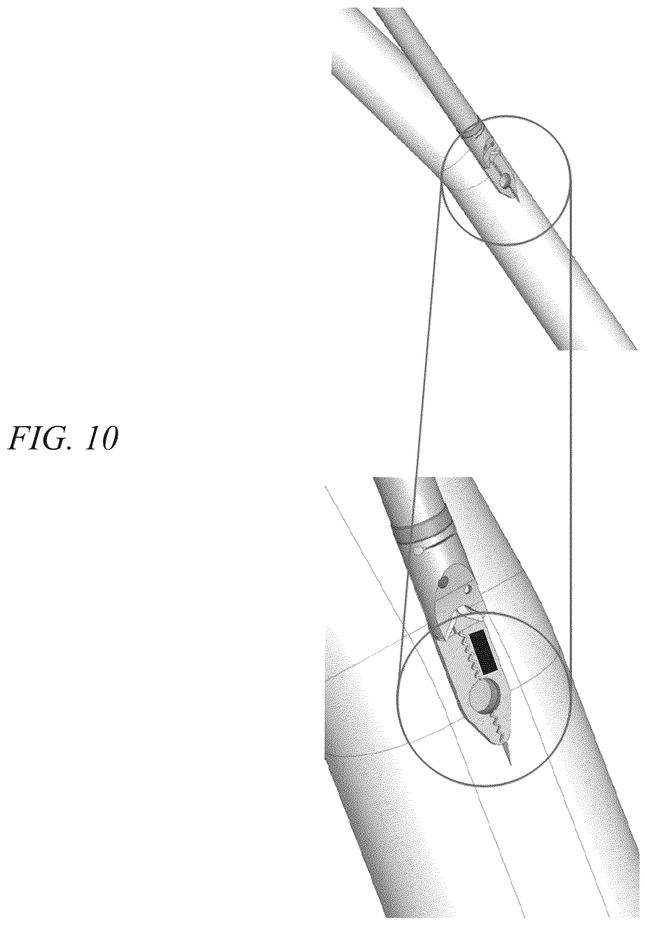

FIGS. 9-12 schematically show various views of a clamp-style apparatus for controlling an acute otomy. The clamp functions by anchoring a periphery of tissue adjacent to the otomy site to reinforce the tissue and distribute the forces applied during subsequent surgical procedures to the apparatus instead of to the tissue. This prevents dilation or contraction of the otomy site. As is shown in FIGS. 9-10, the device contains a rigid or semi-rigid conduit that acts as an open channel to facilitate the deployment and alignment of surgical tools and maintain fluidic passage through the tissue wall. The clamp apparatus may also comprise features to secure compression anastomosis devices to the tool, allowing for one-handed manipulation and control of the tissue and compression anastomosis device.

The clamp apparatus may be deployed endoscopically, laparoscopically, robotically, or in open-field surgery. After the creation of an acute otomy, the clamp clasps onto the tissue surrounding the otomy site. This allows for control of the diameter of the otomy and reduces tissue tearing and dilation around the otomy site.

The clamp may also contain features to secure compression anastomosis devices to the tool, allowing for one-handed manipulation and control of the tissue and compression anastomosis device.

The fenestrated design of various embodiments allows for the clamp to grasp and support the tissue surrounding the otomy site while maintaining an open channel for tool passage during the surgical procedures. In some embodiments, the clamp may remain in place after the magnets are mated to provide a channel for fluidic passage before completion of the permanent anastomosis.

Exemplary embodiments of the clamp-style apparatus may be removed by the medical professional prior to the completion of surgery, leaving behind an otomy or formed anastomosis.

The apparatus of the present invention may be used to facilitate the alignment of an otomy to a mating surgical site to form an anastomosis.

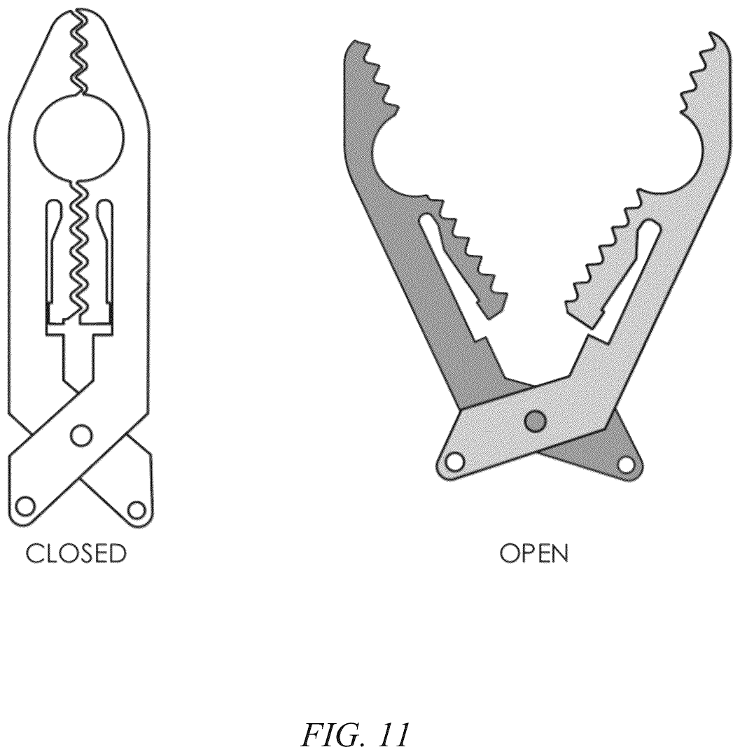

As is shown in FIG. 11, the clamp apparatus may comprise a flexible member or moving hinge point that allows tissue to be clamped proximally before the tissue is clamped distally. This allows for adjustment of tissue between the proximal and distal ends of the grasping element.

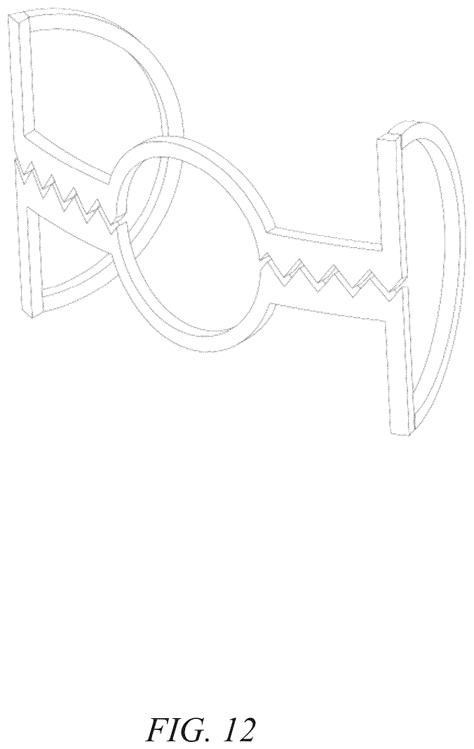

FIG. 12 depicts an exemplary embodiment of a sprung-clamping apparatus or otomy clip. An otomy clip may be deployed around an otomy site to secure tissue. The otomy clip comprises a fenestrated design to allow for fluidic passage through the otomy. The otomy clip clamps the tissue around the otomy site, reducing tearing, dilation, or locomotion of the otomy site.

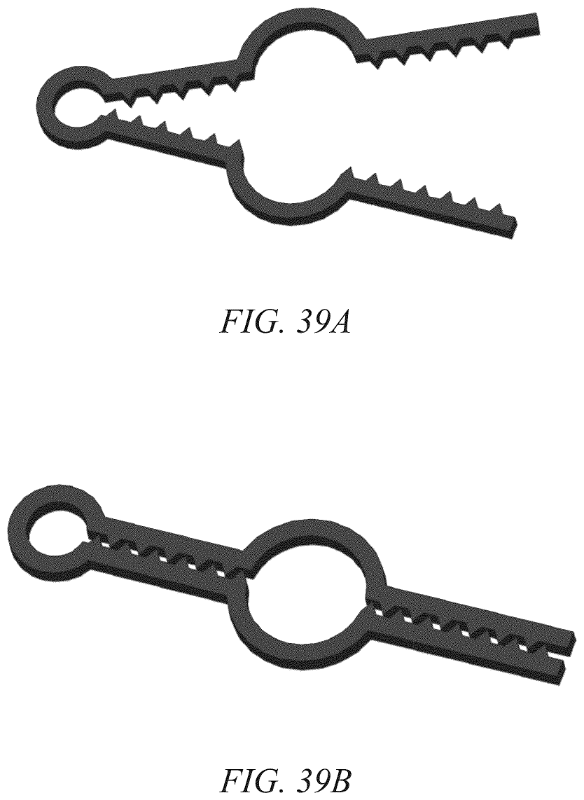

FIGS. 39A-39B depict an embodiment of a sprung clamping otomy clip. An otomy clip may be deployed around an otomy site to secure tissue. The otomy clip comprises a fenestrated design to allow for fluidic passage through the otomy. The otomy clip clamps the tissue around the otomy site, reducing tearing, dilation, or locomotion of the otomy site.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1-14. (canceled)

15. An otomy control device comprising:
a distal flange and a proximal flange separated and connected by a central channel, wherein the distal flange and the proximal flange are configured to compress to fit the otomy control device within a working channel of an access or delivery device in a delivery configuration and to expand on opposite sides of an otomy to secure the otomy when delivered from the working channel in a fully deployed configuration, further comprising at least one magnet, wherein the at least one magnet is situated about the central channel.

16-22. (canceled)

23. An otomy control device comprising:
a distal flange and a proximal flange separated and connected by a central channel, wherein the distal flange and the proximal flange are configured to compress to fit the otomy control device within a working channel of an access or delivery device in a delivery configuration and to expand on opposite sides of an otomy to secure the otomy when delivered from the working channel in a fully deployed configuration, wherein the otomy control device is a two-piece device comprising a distal half including the distal flange and a proximal half including the proximal flange, wherein the distal half and the proximal half are configured to interlock to form the central channel, further comprising at least one connecting member to assist with mating of the device with another otomy control device.

24-25. (canceled)
